(19) United States
(12) Patent Application Publication
(10) Pub. No.: US 2026/0259184 A1
(43) Pub. Date: Sep. 3, 2026

(54) METHODS OF PERFORMING HIGH-FLOW CHROMATOGRAPHY UNDER ADIABATIC CONDITIONS

(71) Applicant: Waters Technologies Corporation, Milford, MA (US)

(72) Inventors: Fabrice Gritti, Franklin, MA (US); Joseph A. Jarrell, Newton Highlands, MA (US)

(73) Assignee: Waters Technologies Corporation, Milford, MA (US)

(21) Appl. No.: 19/551,201

(22) Filed: Feb. 26, 2026

Related U.S. Application Data
(60) Provisional application No. 63/764,992, filed on Feb. 28, 2025.

(51) Int. Cl. G01N 30/86; C12N 15/10; G01N 30/02; G01N 30/66; G01N 30/88

(52) U.S. Cl. CPC G01N 30/8631; C12N 15/101; G01N 30/66; G01N 2030/027; G01N 2030/8827

(57) ABSTRACT

The present disclosure provides for methods of separating nucleic acid species by size under high-flow and adiabatic conditions. Typically, nucleic acids are separated based on electrophoretic mobility using gel electrophoresis or slalom chromatography allows for rapid separation of nucleic acid species by size. Gel electrophoresis methods suffer from low yields and long time scales for performing the characterizations. Slalom chromatography, on the other hand, utilizes high pressure conditions which results in friction being generated between the analyte, the chromatography material, and the mobile phase, resulting in frictional heating and temperature gradients throughout the column. These gradients typically work against any performance gains obtained from the high-flow conditions.

### CROSS-REFERENCE TO RELATED APPLICATION

This application claims priority from and the benefit of U.S. Application No. 63/764,992 filed on Feb. 28, 2025. The entire contents of this application are incorporated herein by reference.

### FIELD OF INVENTION

The present disclosure relates generally to methods of separating nucleic acids using liquid chromatography, particularly slalom chromatography.

### BACKGROUND

Nucleic acids form the basis of molecular cloning techniques, are critical in the manufacture of biologic therapeutics such as antibodies and are at the forefront of new modalities such as cell/gene therapies and ARNA-based vaccines. As such, the ability to manipulate, separate, and characterize nucleic acids is foundational across academic research, biotechnology, and pharmaceutical industries.

Accordingly, there is a need for methods of performing high-flow chromatography under conditions which reduce thermal gradients throughout the chromatography column.

### SUMMARY OF INVENTION

The present disclosure provides for methods of separating nucleic acid species by size under high-flow and adiabatic conditions. Typically, nucleic acids are separated based on electrophoretic mobility using gel electrophoresis or slalom chromatography, which allows for rapid separation of nucleic acid species by size. Gel electrophoresis methods suffer from low yields and long time scales for performing the characterizations. Slalom chromatography, on the other hand, utilizes high pressure conditions which results in friction being generated between the analyte, the chromatography material, and the mobile phase, resulting in frictional heating and temperature gradients throughout the column. These gradients typically work against any performance gains obtained from the high-flow conditions.

Accordingly, in one aspect, disclosed herein is a method of separating a plurality of oligonucleotides, the method including:
(a) loading a sample comprising the plurality of oligonucleotides, the plurality of nucleotides including a plurality of a first oligonucleotide and a plurality of a second oligonucleotide, onto a chromatography system;
   wherein the chromatography system comprises a chromatography column, a jacket surrounding the liquid chromatography column, and a vacuum chamber formed in an area between the liquid chromatography column and the jacket;
   wherein the chromatography column comprises a column body and a plurality of particles, the column body comprising an inlet region, an outlet region, an interior surface, and an exterior surface;
(b) eluting the sample from the chromatography column by contacting the chromatography column with a mobile phase;
   wherein the chromatography column is characterized by a flow rate and a pressure differential between the inlet region and the outlet region, wherein the product of the flow rate and the pressure differential is at least 1.5 W/m; and
(c) detecting the plurality of the first oligonucleotide and/or the plurality of the second oligonucleotide in the eluent, wherein the plurality of the first oligonucleotide form at least one distinct peak and wherein the plurality of the second oligonucleotide form at least one distinct peak;
wherein the chromatography column is characterized by a radial temperature gradient of at most 0.1 K and a pressure differential from the inlet region of the chromatography column to the outlet region of the chromatography column of at least 100 bar.

In some embodiments, the first oligonucleotide comprises DNA or RNA. In some embodiments, the DNA comprises single stranded DNA (ssDNA) or double stranded DNA (dsDNA). In some embodiments, the dsDNA is linear, circular, looped, or supercoiled. In some embodiments, the RNA comprises single stranded RNA (ssRNA) or double stranded RNA (dsRNA). In some embodiments, the ssRNA is linear, looped, or circular.

In some embodiments, the first oligonucleotide comprises ssRNA, and the second oligonucleotide comprises dsDNA. In some embodiments, the first oligonucleotide comprises ssDNA, and the second oligonucleotide comprises dsDNA. In some embodiments, the first oligonucleotide comprises ssRNA, and the second oligonucleotide comprises dsRNA.

In some embodiments, the first oligonucleotide comprises from about 1000 to about 100000 base pairs. In some embodiments, the second oligonucleotide comprises from about 1000 to about 100000 base pairs.

In a second aspect, disclosed herein is a method of separating DNA and RNA, the method comprising:
(a) loading a sample comprising a plurality of ssRNA and a plurality of dsDNA molecules onto a chromatography system;
   wherein the chromatography system comprises a chromatography column, a jacket surrounding the liquid chromatography column, and a vacuum chamber formed in an area between the liquid chromatography column and the jacket;
   wherein the chromatography column comprises a column body and a plurality of particles, the column body comprising an inlet region, an outlet region, an interior surface, and an exterior surface;
(b) eluting the sample from the chromatography column by contacting the chromatography column with a mobile phase;
   wherein the chromatography column is characterized by a flow rate and a pressure differential between the inlet region and the outlet region, wherein the product of the flow rate and the pressure differential is at least 1.5 W/m; and
(c) detecting the plurality of ssRNA and/or the plurality of dsDNA in the eluent, wherein the plurality of ssRNA form at least one distinct peak and wherein the plurality of dsDNA form at least one distinct peak;
wherein the chromatography column is characterized by a radial temperature gradient of at most 0.1 K and a pressure differential from the inlet region to the outlet region of at least 100 bar.

In a third aspect, disclosed herein is a method of separating dsDNA, the method comprising:
(a) loading a sample comprising a plurality of dsDNA molecules onto a chromatography system;
   wherein the chromatography system comprises a chromatography column, a jacket surrounding the liquid chromatography column, and a vacuum chamber formed in an area between the liquid chromatography column and the jacket;
   wherein the chromatography column comprises a column body and a plurality of particles, the column body comprising an inlet region, an outlet region, an interior surface, and an exterior surface;
(b) eluting the sample from the chromatography column by contacting the chromatography column with a mobile phase;
   wherein the chromatography column is characterized by a flow rate and a pressure differential between the inlet region and the outlet region, wherein the product of the flow rate and the pressure differential is at least 1.5 W/m; and
(c) detecting the plurality of dsDNA in the eluent, wherein the plurality of dsDNA form at least two distinct peaks;
wherein the chromatography column is characterized by a radial temperature gradient of at most 0.1 K and a pressure differential from the inlet region to the outlet region of at least 100 bar.

In some embodiments of the third aspect, the dsDNA is linear, circular, looped, or supercoiled. In some embodiments, the sample further comprises a plurality of ssRNA molecules, a plurality of dsRNA molecules, and/or a plurality of ssDNA molecules. In some embodiments, the sample comprises a plurality of ssRNA molecules, wherein the ssRNA is linear, looped, or circular.

In a fourth aspect, disclosed herein is a method of separating DNA, the method comprising:
(a) loading a sample comprising a plurality of dsDNA molecules and a plurality of ssDNA molecules onto a chromatography system;
   wherein the chromatography system comprises a chromatography column, a jacket surrounding the liquid chromatography column, and a vacuum chamber formed in an area between the liquid chromatography column and the jacket;
   wherein the chromatography column comprises a column body and a plurality of particles, the column body comprising an inlet region, an outlet region, an interior surface, and an exterior surface;
(b) eluting the sample from the chromatography column by contacting the chromatography column with a mobile phase;
   wherein the chromatography column is characterized by a flow rate and a pressure differential between the inlet region and the outlet region, wherein the product of the flow rate and the pressure differential is at least 1.5 W/m; and
(c) detecting the plurality of dsDNA and/or the plurality of ssDNA in the eluent, wherein the plurality of dsDNA forms at least one distinct peak, and the plurality of ssDNA forms at least one distinct peak;
wherein the chromatography column is characterized by a radial temperature gradient of at most 0.1 K and a pressure differential from the inlet region to the outlet region of at least 100 bar.

In a fifth aspect, disclosed herein is a method of separating RNA, the method comprising:
(a) loading a sample comprising a plurality of dsRNA molecules and a plurality of ssRNA molecules onto a chromatography system;
   wherein the chromatography system comprises a chromatography column, a jacket surrounding the liquid chromatography column, and a vacuum chamber formed in an area between the liquid chromatography column and the jacket;
   wherein the chromatography column comprises a column body and a plurality of particles, the column body comprising an inlet region, an outlet region, an interior surface, and an exterior surface;
(b) eluting the sample from the chromatography column by contacting the chromatography column with a mobile phase;
   wherein the chromatography column is characterized by a flow rate and a pressure differential between the inlet region and the outlet region, wherein the product of the flow rate and the pressure differential is at least 1.5 W/m; and
(c) detecting the plurality of dsRNA and/or the plurality of ssRNA in the eluent, wherein the plurality of dsRNA forms at least one distinct peak, and the plurality of ssRNA forms at least one distinct peak;
wherein the chromatography column is characterized by a radial temperature gradient of at most 0.1 K and a pressure differential from the inlet region to the outlet region of at least 100 bar.

In some embodiments of any preceding aspect, the mobile phase comprises water and one or more salts; wherein the mobile phase does not comprise acetonitrile. In some embodiments, the mobile phase comprises water, acetonitrile, and one or more salts. In some embodiments, the concentration of acetonitrile in the mobile phase is not sufficient to denature the oligonucleotide, DNA, or RNA. In some embodiments, the acetonitrile is less than 30%/w of the mobile phase.

In some embodiments, the concentration of the one or more salts is tailored to result in a zone retention factor (k₁) of less than 0.35 for at least one of the oligonucleotides.

In some embodiments, the eluting in step (b) is characterized by a k₁ of less than 0.35 for at least one of the oligonucleotides of the sample. In some embodiments, the eluting in step (b) is characterized by a k₁ of from −0.2 to 0.35 for at least one of the oligonucleotides of the sample. In some embodiments, the eluting in step (b) is characterized by a k₁ of about 0 for at least one of the oligonucleotides of the sample. In some embodiments, all oligonucleotides of the sample are characterized by a k₁ of less than 0.35. In some embodiments, all oligonucleotides of the sample are characterized by a k₁ of from −0.2 to 0.35. In some embodiments, the k₁ of all oligonucleotides of the sample is about 0.

In some embodiments, the eluting in step (b) is performed in from about 0.6 minutes to about 10 minutes. In some embodiments, the eluting in step (b) is performed in from about 1 minute to about 3 minutes. In some embodiments, the eluent is collected.

In some embodiments, the plurality of particles comprise an organic material. In some embodiments, the plurality of particles comprise a material with a thermal conductivity of at most 2 W/m·K (e.g., a thermal conductivity of at most 1.4 W/m·K). In some embodiments, the chromatography system is characterized by an effective thermal conductivity of at most 2 W/m·K (e.g., at most 1.4 W/m·K). In some embodiments, the effective thermal conductivity of the chromatography system is from about 0.2 W/m·K to about 1.4 W/m·K. In some embodiments, the plurality of particles comprise an average pore diameter from about 20 Å to about 900 Å. In some embodiments, the average pore diameter of the plurality of porous particles is about 125 Å. In some embodiments, the average pore diameter of the plurality of porous particles is about 250 Å. In some embodiments, the average specific pore volume of the plurality of particles is from about 0.5 cm³/g to about 1.3 cm³/g.

In some embodiments, the plurality of particles have an average diameter of from 1 μm to about 10 μm. In some embodiments, the plurality of particles have an average diameter of about 1.7 μm. In some embodiments, the plurality of particles have an average diameter of about 2.5 μm.

In some embodiments, the chromatography column comprises a length measured form a proximal end of the inlet region to a distal end of the outlet region, wherein the length is from about 5 cm to about 50 cm. In some embodiments, the length is about 15 cm. In some embodiments, the length is about 30 cm.

In some embodiments, the average diameter of the plurality of particles is about 1.7 μm; and the chromatography column comprises a length measured form a proximal end of the inlet region to a distal end of the outlet region, wherein the length is about 15 cm.

In some embodiments, the average diameter of the plurality of particles is about 2.5 μm; and the chromatography column comprises a length measured form a proximal end of the inlet region to a distal end of the outlet region, wherein the length is about 30 cm.

In some embodiments, the elution of step (b) is performed at a flow rate of at least 0.50 mL/min. In some embodiments, the elution of step (b) is performed at a flow rate of at least 0.75 mL/min. In some embodiments, the elution of step (b) is performed at a flow rate of at least 1.0 mL/min.

In some embodiments, at least a portion of the interior surface of the chromatography column body is coated with an alkylsilyl material. In some embodiments, the alkylsilyl material is hydrophilic. In some embodiments, the alkylsilyl material is non-ionic. In some embodiments, the alkylsilyl material is polyethylene glycol silane. In some embodiments, the alkylsilyl material is a product of vapor deposition of bis(trichlorosilyl)ethane and bis(trimethoxylsilyl)ethane.

In some embodiments, the vacuum chamber comprises a thermal conductivity of at most 0.2 W/m·K. In some embodiments, the vacuum chamber comprises a pressure of at most 1 mbar. In some embodiments, the vacuum chamber comprises a pressure of at most 0.001 mbar. In some embodiments, the vacuum chamber comprises an inert gas, the inert gas selected from argon, krypton, xenon, carbon dioxide, and sulfur hexafluoride. In some embodiments, the vacuum chamber comprises an opening characterized by a width in fluid communication with a vacuum pump, wherein the inert gas is characterized by a mean free path, and the mean free path of the inert gas is at least 100 times larger (e.g., at least 1000 times larger) than the width of the opening.

In some embodiments, the chromatography system further comprises one or more temperature sensors, each temperature sensor configured to sense a temperature along a different portion of a length of the chromatography column. In some embodiments, the chromatography system further comprises a plurality of temperature sensors. In some embodiments, the plurality of temperature sensors comprises an inlet temperature sensor configured to determine the temperature of the inlet region and an outlet temperature sensor configured to determine the temperature of the outlet region. In some embodiments, the chromatography system further comprises at least one processor operative to execute the computer-executable code to cause the apparatus to determine the temperature of the liquid chromatography column from the plurality of temperature sensors, and individual control at least one of the temperature sensors based on the inlet temperature or the outlet temperature.

In some embodiments, the at least distinct peak is characterized by an increase in the USP plate count compared to a chromatography column not including a vacuum jacket and/or vacuum chamber. In some embodiments, the increase in USP plate count is at least 10%. In some embodiments, the increase in USP plate count is at least 20%. In some embodiments, the increase in USP plate count is at least 30%.

In some embodiments, the at least distinct peak is characterized by a decrease in full-width-at-half-max compared to a chromatography column not including a vacuum jacket and/or vacuum chamber. In some embodiments, the decrease in full-width-at-half-max is at least 10%. In some embodiments, the decrease in full-width-at-half-max is at least 20%. In some embodiments, the decrease in full-width-at-half-max is at least 30%.

### Definitions

Disclosed herein are methods and systems for the separation of nucleic acids under high-flow conditions (i.e., slalom chromatography). In particular, the present disclosure relates to methods of separating nucleic acids under high pressure without forming a thermal gradient. In order that the technology may be more readily understood, certain terms are first defined. It should be noted that whenever a value or range of values of a parameter are recited, it is intended that values and ranges intermediate to the recited values are part of this disclosure. The word “about” if not otherwise defined means ±5%. It is also to be noted that as used herein and in the claims, singular forms of “a”, “an”, and “the” include plural references unless the context clearly dictates otherwise. Additional terms are defined throughout the specification.

As used herein, the term “slalom chromatography” refers to a chromatographic method performed under high-flow conditions that separates a molecule by size based on hydrodynamic phenomena. The principles of slalom chromatography are further described in Hirabayashi and Kasai, J. Chromatogr. A (1996) 722(1-2):135-42 and Hirabayashi et al., Biochemistry (1990) 29(41):9515-21. The high-flow rates used in slalom chromatography (SC) afford sufficiently high shear rates that result in prolonged extension of the molecules, e.g., nucleic acids, which is important for the separation of the molecules by size. The extension of the molecule can be assessed using the Weissenberg number.

As used herein, the term “nucleic acids” or “nucleic acid molecules” refers to a polymeric molecule comprising two or more nucleotides. Nucleic acids may comprise deoxyribonucleic acids (DNA), ribonucleic acids (RNA), or a combination thereof. DNA comprises the nucleotides cytidine, guanosine, adenosine, and thymidine. RNA comprises the nucleotides cytidine, guanosine, adenosine, and uridine. In some embodiments, the nucleic acid molecules may comprise nucleic acid analogues (i.e., non-naturally occurring nucleic acids or analogues thereof). Examples of nucleic acid analogues include peptide nucleic acids, locked nucleic acids, glycol nucleic acids, threose nucleic acids, hexitol nucleic acids. Nucleic acid analogues are further reviewed in Wang et al., Molecules (2023) 28(20):7043. Nucleic acid molecules may further be modified at the nucleobase, sugar, or phosphodiester backbone with an array of chemical modifications which are further reviewed in Epple et al., Emerg. Top. Life. Sci. (2021) 5(5):691-697.

Nucleic acid molecules range in length as measured by the number of nucleotides or base pairs. In embodiments of the present technology, the nucleic acids may range from about 5000 base pairs to about 100,000 base pairs. Nucleic acids may be single stranded (e.g., single-stranded DNA (ssDNA) or single-stranded RNA (ssRNA)). In some embodiments, the ssRNA is mRNA. Nucleic acids may be double stranded (e.g., double-stranded DNA (dsDNA) or double-stranded RNA (dsRNA). Double-stranded nucleic acids are made of complementary sequences (e.g., base-paired sequences) as is known in the art and would be readily understood by one of ordinary skill.

Nucleic acids may be present in different topologies, including linear and circular conformations. In some embodiments, circular nucleic acids may be supercoiled, in which the circular nucleic acid molecule undergoes additional twist strain.

As used herein, the term “porous” refers to a material that has a pore volume that is greater than 0.1 cc/g. Preferably, porous polymers have a pore volume that is greater than 0.1 cc/g (e.g., 0.5 cc/g). As used herein, the term “non-porous” refers to a material that has a pore volume that is less than 0.1 cc/g. As used herein, the term “superficially porous” refers to a material having a non-porous region (e.g., a core) and a porous region (e.g., a shell). Preferably, non-porous materials (or the non-porous region of a material) have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

As used herein, the term “superficially porous particles” refers to a material that has a solid or non-porous core (solid-core) and an outer layer surrounding the core that is porous or has a greater degree of porosity that the non-porous core.

As used herein, the term “zone retention factor,” also referred to as k₁, refers to the ratio of retention of an analyte (e.g., a nucleic acid molecule) on a column and the void retention volume. A plot of the zone retention factor k₁ of an analyte as a function of the shear stress variable r corresponding to that analyte may be used to empirically determine the k₁ value.

As used herein, the term “distinct peak” refers to a peak in a chromatogram, e.g., a detected analyte, that is baseline resolved. A chromatography experiment resulting in two or more distinct peaks refers to a chromatogram in which a first baseline resolved distinct peak is resolvable from a second baseline resolved distinct peak. The methods of the present disclosure may allow for the resolution of two or more distinct peaks in a chromatogram, each distinct peak corresponding to a unique detected analyte.

As used herein, the term “selectivity factor” or “a” refers to the ability of a chromatographic method or system to distinguish analytes within a sample, typically a ratio of two peaks.

As used herein, “radial temperature gradient” refers to the difference in temperature between two points in a chromatography column. The two points of the radial temperature gradient may be, e.g., the center of a chromatography column, and a wall of the chromatography column body. In preferred embodiments, the radial temperature gradient is defined along a cross sectional area perpendicular to the length of the chromatography column. In methods of the present disclosure, a radial temperature gradient may be defined at multiple points along a chromatography column. For example, a chromatography column including an inlet region, a middle region, and an outlet region may be characterized by a radial temperature gradient at the inlet region, a radial temperature gradient at the middle region, and a radial temperature gradient at the outlet region. Methods of the present disclosure may provide for chromatography columns characterized by a radial temperature gradient of at most 0.1 K (e.g., at most 0.05 K, at most 0.01 K, at most 0.005 K, at most 0.001 K, etc.) at all cross sectional areas along the length of the chromatography column.

The separation of nucleic acids using the described methods can be achieved with high resolution by manipulating, for example, the particle size, column length, and column pressure. By adjusting said variables according to the teachings described herein and as shown in the examples (see, e.g., Example 4), maximum resolution can be achieved for DNA varying in nucleotide length.

### BRIEF DESCRIPTION OF THE DRAWINGS

The technology will be more fully understood from the following detailed description taking in conjunction with the accompanying drawings.

FIG. 1 is a graphical illustration of a method of separating nucleic acid molecules according to some embodiments of the present technology.

FIG. 2 provides a chromatograph of double-stranded DNA species separated according to some embodiments of the present technology.

FIG. 3 provides a chromatograph of circular and linear DNA species separated according to some embodiments of the present technology.

FIG. 4 provides a chromatograph of single-stranded and double-stranded RNA separated according to some embodiments of the present technology.

FIG. 5A-5H provides chromatographs of double-stranded DNA separated using mobile phases of varying ionic strengths. FIG. 5A shows a chromatograph obtained at a PBS concentration of 0.001 fold. FIG. 5B shows a chromatograph obtained at a PBS concentration of 0.001 fold. FIG. 5C shows a chromatograph obtained at a PBS concentration of 0.01 fold. FIG. 5D shows a chromatograph obtained at a PBS concentration of 0.01 fold. FIG. 5E shows a chromatograph obtained at a PBS concentration of 0.1 fold. FIG. 5F shows a chromatograph obtained at a PBS concentration of 0.1 fold. FIG. 5G shows a chromatograph obtained at a PBS concentration of 1 fold. FIG. 5H shows a chromatograph obtained at a PBS concentration of 1 fold.

FIG. 6A-6B show chromatographs of dsRNA at different shear rates. FIG. 6A shows a chromatograph taken at a low shear rate of 0.025 mL/min. FIG. 6B shows a chromatography taken at a high shear rate of 1.2 mL/min. The dotted lines between FIG. 6A and FIG. 6B correspond to the peak shift between the experiments.

FIG. 7A-7B provide chromatographs of double-stranded DNA samples separated at various flow rates. FIG. 7A provides chromatographs of supercoiled dsDNA separated from linear dsDNA.

FIG. 7B provides chromatographs of circular dsDNA separated from linear dsDNA.

FIG. 8A-8B demonstrate the correlation between nucleic acid length and k coefficients of dsDNA separated using methods according to some embodiments of the present technology.

FIG. 9 provides a chromatograph of a DNA and RNA mixture separated according to embodiments of the present technology.

FIG. 10 provides a chromatograph of a DNA digest separated according to some embodiments of the technology.

FIG. 11 provides a chromatograph of a small interfering RNA (siRNA) calibrant separated according to some embodiments of the technology and detected using multi-angle light scattering (MALS).

FIGS. 12A-12F provide chromatographs of a DNA digest separated according to some embodiments of the technology and detected using multi-angle light scattering (MALS).

FIG. 12A provides the chromatograph before UV broadening.

FIG. 12B provides the chromatograph after UV broadening.

FIG. 12C provides Zimm plots for the first of two peaks as detected by MALS.

FIG. 12D provides Zimm plots for the second of two peaks as detected by MALS.

FIG. 12E provides Random Coil plots for the first of two peaks as detected by MALS.

FIG. 12F provides Random Coil plots for the second of two peaks as detected by MALS.

FIG. 13 provides a chromatograph of a DNA digest at concentrations ranging from 0.1 μg to 5 μg separated according to some embodiments of the technology and detected using MALS.

FIG. 14 provides a representative chromatograph of a DNA digest separated using methods according to the present technology and detected using multi-angle light scattering (MALS) and dynamic light scattering (DLS) detectors.

FIG. 15 provides representative chromatographs for the separation of DNA from additional mixture components using a particle having a 125 Å pore size (top) or a 250 Å pore size (bottom).

FIG. 16 provides a chromatograph showing the theoretical separation of dsRNA and ssRNA present in a mixture relative to standard nucleic acid ladders.

FIG. 17 provides an exemplary graph depicting the resolution of nucleic acid species separations according to embodiments of the technology.

FIG. 18A-18D provide thermal plots displaying the axial thermal gradient along an exemplary chromatography column in a diabatic environment. FIG. 18A shows a thermal plot at the inlet region of the chromatography column. FIG. 18B shows a thermal plot at a middle region of the chromatography column. FIG. 18C shows a thermal plot at the outlet region of the chromatography column. FIG. 18D shows a plot of the temperature at various points along a length of the chromatography column.

FIG. 19A-19C provide plots depicting the radial temperature gradient of an exemplary chromatography column in a diabatic environment. FIG. 19A shows the radial temperature gradient near the inlet region of the chromatography column. FIG. 19B shows the radial temperature gradient near the middle region of the chromatography column. FIG. 19C shows the radial temperature gradient near the outlet region of the chromatography column.

FIG. 20 provides a 3D plot depicting the temperature gradient along the length and width of an exemplary chromatography column in a diabatic environment.

FIG. 21 provides a plot comparing the radial thermal gradient of a diabatic chromatography column (circles) and a chromatography column of the present technology (squares).

FIG. 22 provides two chromatograms depicting the resolution of nucleic acid species separations using a BEH Premier Protein SEC (250 Å) 4.6×300 mm column, using a 100 mM phosphate buffer at pH 8 and a flow rate of 1.0 mL/min. The chromatogram on the left shows an exemplary separation performed under adiabatic conditions. The chromatogram on the right shows an exemplary separation performed under diabatic conditions. The gel electrophoresis experiment displayed in the center shows the weights of the DNA oligonucleotides in the sample.

FIG. 23 provides two chromatograms depicting the resolution of nucleic acid species separations using BEH Premier Protein SEC (250 Å) 4.6×300 mm column, using a 100 mM phosphate buffer at pH 8 and a flow rate of 1.0 mL/min. The chromatogram on the left shows an exemplary separation performed under adiabatic conditions. The chromatogram on the right shows an exemplary separation performed under diabatic conditions. The gel electrophoresis experiment displayed in the center shows the weights of the DNA oligonucleotides in the sample.

FIG. 24 provides two chromatograms depicting the resolution of nucleic acid species separations using a 4.6×150 mm column having a pore size of (90 Å), using a 100 mM phosphate buffer at pH8 and a flow rate of 1.7 mL/min. The chromatogram on the left shows an exemplary separation performed under adiabatic conditions. The chromatogram on the right shows an exemplary separation performed under diabatic conditions. The gel electrophoresis experiment displayed in the center shows the weights of the DNA oligonucleotides in the sample.

FIG. 25 provides two chromatograms depicting the resolution of nucleic acid species separations using a 4.6×150 mm column having a pore size of (90 Å), using a 100 mM phosphate buffer at pH 8 and a flow rate of 1.7 mL/min. The chromatogram on the left shows an exemplary separation performed under adiabatic conditions. The chromatogram on the right shows an exemplary separation performed under diabatic conditions. The gel electrophoresis experiment displayed in the center shows the weights of the DNA oligonucleotides in the sample.

FIG. 26 provides two chromatograms depicting the resolution of nucleic acid species separations using a 4.6×150 mm column having a pore size of (120 Å), using a 100 mM phosphate buffer with 20% by volume acetonitrile at pH 8 and a flow rate of 1.7 mL/min. The chromatogram on the left shows an exemplary separation performed under adiabatic conditions. The chromatogram on the right shows an exemplary separation performed under diabatic conditions. The gel electrophoresis experiment displayed in the center shows the weights of the DNA oligonucleotides in the sample.

FIG. 27 provides two chromatograms depicting the resolution of nucleic acid species separations using a 4.6×150 mm column having a pore size of (120 Å), using a 100 mM phosphate buffer with 20% by volume acetonitrile at pH 8 and a flow rate of 1.7 mL/min. The chromatogram on the left shows an exemplary separation performed under adiabatic conditions. The chromatogram on the right shows an exemplary separation performed under diabatic conditions. The gel electrophoresis experiment displayed in the center shows the weights of the DNA oligonucleotides in the sample.

FIG. 28 provides two chromatograms depicting the resolution of nucleic acid species separations using a 4.6×150 mm column having a pore size of (90 Å), using a 100 mM phosphate buffer at pH 7.4 and a flow rate of 1.2 mL/min. The chromatogram on the left shows an exemplary separation performed under adiabatic conditions. The chromatogram on the right shows an exemplary separation performed under diabatic conditions. The gel electrophoresis experiment displayed in the center shows the weights of the DNA oligonucleotides in the sample.

FIG. 29 provides two chromatograms depicting the resolution of nucleic acid species separations using a 4.6×150 mm column having a pore size of (90 Å), using a 100 mM phosphate buffer at pH 7.4 and a flow rate of 1.2 mL/min. The chromatogram on the left shows an exemplary separation performed under adiabatic conditions. The chromatogram on the right shows an exemplary separation performed under diabatic conditions. The gel electrophoresis experiment displayed in the center shows the weights of the DNA oligonucleotides in the sample.

### DETAILED DESCRIPTION

The present disclosure is related to methods of separating nucleic acid species by size under high-flow and adiabatic conditions. Typically, nucleic acids are separated based on electrophoretic mobility using gel electrophoresis or analogous methods. These approaches can be time intensive (ranging from multiple hours to days) and are not sensitive enough to detect nucleic acid heterogeneity, and hinder downstream processing of samples due to the need to extract nucleic acids from the gel itself, substantially lowering yield and increased contaminants. To circumvent the limitations of gel electrophoresis, practitioners often turn to high-flow chromatography, often referred to as slalom chromatography. Slalom chromatography allows for rapid separation of nucleic acid species by size. As these methods utilize high performance liquid chromatography (HPLC) or ultra-high performance liquid chromatography (UHPLC) systems, the methods afford the separation and characterization of complex mixtures of nucleic acid species.

Because of the high pressures utilized in slalom chromatography, friction is generated between the analyte, the chromatography material, and the mobile phase. The friction results in frictional heating, which in turn leads to the formation of temperature gradients throughout the column. These gradients typically work against any performance gains obtained from the high-flow conditions.

The present disclosure relates to methods of performing high-flow chromatography under adiabatic conditions. The disclosed methods are capable of separating nucleic acids by size in under 10 minutes with high efficiency.

### High-Flow Chromatography

The methods disclosed herein utilize conditions that result in a high shear rate such that the nucleic acid molecules in the column are extended/elongated under the shear flow. Extension or elongation of nucleic acid molecules can be determined by the Weissenberg number, which compares elastic and viscous forces exerted on a molecule. The Weissenberg number is a dimensionless metric that is a function of relaxation time and shear rate:

$$Wi = {τ}_{R} \dot{γ}$$

wherein τ_{R} is the relaxation time seconds) as represented by the formula:

$${τ}_{R} = \frac{{nL}_{c}^{2} {I}_{P}}{{k}_{B} T}$$

wherein h is dynamic velocity (Pa*s); Lc is contour length (m); I_{P} is persistence length (m); k_{B} is the Boltzmann constant (1.38×10⁻²³ J·K⁻¹), and T is temperature (Kelvin); and
\dot{γ} is shear rate (seconds⁻¹) as represented by the formula:

$$\dot{γ} = \frac{Δ u}{Δ z} = \frac{16}{3} < u > / d$$

wherein Δu is the velocity difference between two points in a direction perpendicular to the flow direction, the two points separated by a distance of Δz; <u> is the average linear interstitial velocity along the packed bed; and
d is the average flow-through tube diameter.

The relaxation time τ_{R} is dependent, in part, on intrinsic properties of the target nucleic acid. In certain instances, the relaxation time of a molecule under certain conditions is known in the art. For example, the Lc of linear dsDNA is equal to the number of base pairs*3.4 Å; the dynamic velocity of linear dsDNA is 1.2 cP; and the persistence length of linear dsDNA is 450 Å. Alternatively, relaxation time of a molecule under certain conditions may be determined empirically using known methods that are readily understood by one of ordinary skill in the art. (see e.g., Bouchiat et al., Biophysical Journal (1999) 76:409-413).

In instances wherein the Weissenberg number is greater than 1, the nucleic acid molecule is extensible under the shear flow and would be separated by size based on the principles of slalom chromatography. In instances wherein the Weissenberg number is less than 1, the nucleic acid molecule is not extensible under the shear flow and would not be separated by size based on the principles of slalom chromatography.

Accordingly, in one aspect the methods disclosed herein utilize conditions that result in the nucleic acid molecules exhibiting a Weissenberg number (Wi) of greater than 1. In some embodiments, the Wi is between 1-100, 5-100, 10-100, 15-100, 20-100, 25-100, 30-100, 35-100, 40-100, 45-100, 55-100, 60-100, 65-100, 70-100 75-100, 80-100, 85-100, 90-100, or 95-100.

Shear rate, and consequently the Weissenberg number (Wi), can be influenced by several parameters, including flow rate, particle size, mobile phase viscosity, and temperature. Thus, said variables may be adjusted to achieve sufficient Wi numbers for the separation of the desired nucleic acid molecules.

### Adiabatic Chromatography

Methods of the present invention highlight adiabatically isolating the liquid chromatography column. When a sample flows through the chromatography system at high pressure, frictional heat is generated within the column. The amount of frictional heat generated is a function of several factors, including the flow rate of the mobile phase, the particle size of the column packing material, the dimensions (e.g., length and inner diameter) of the liquid chromatography column, or the properties of the mobile phase (e.g., the thermal conductivity of the mobile phase solvent). Because frictional heat results from interaction with the chromatography material, more heat is generated in areas of the column with more contact with the chromatography material (e.g., the center of the chromatography column as compared with an edge of the chromatography column or the outlet of the chromatography column as compared with the inlet of the chromatography column). These differences in frictional heat in turn produce thermal gradient along the length of the chromatography column (i.e., axial temperature gradients) or the width of the chromatography column (i.e., radial temperature gradients). Because many critical factors governing the performance of chromatography columns (e.g., the viscosity of the mobile phase solvent, analyte retention, or elongation of the analyte) are temperature dependent, temperature differences within a chromatography column (particularly radial temperature differences) increase the dispersion of analytes within a chromatography column. This in turn reduces chromatography column performance (e.g., by broadening chromatogram peaks).

Without wishing to be bound by theory, the radial temperature gradient (ΔT) of a chromatography column may be calculated through Equation 1 below:

$$Δ T = ( 1 + {α}_{P} T ) \frac{{F}_{ν} Δ P}{4 π {λ}_{P} L} ; \qquad Equation 1$$

wherein α_{P} is the thermal expansion coefficient of the mobile phase;
λ_{P} is the thermal conductivity of the chromatography material;
T is the absolute temperature of the chromatography system;
Fᵥ is the flow rate;
ΔP is the pressure differential across the chromatography column; and
L is the length of the chromatography column.

In some embodiments, methods of the present invention reduce radial temperature gradients to at most 1 K (e.g., at most 0.9 K, at most 0.8 K, at most 0.7 K, at most 0.6 K, at most 0.5 K, at most 0.4 K, at most 0.3 K, at most 0.2 K, at most 0.1 K, at most 0.09 K, at most 0.08 K, at most 0.07 K, at most 0.06 K, at most 0.05 K, at most 0.04 K, at most 0.02 K, at most 0.01 K, at most 0.005 K, at most 0.001 K, etc). In some embodiments, methods of the present disclosure are capable of providing a pressure differential of at least 50 bar (e.g., at least 100 bar, at least 150 bar, at least 200 bar, at least 250 bar, at least 300 bar, at least 350 bar, at least 400 bar, at least 450 bar, at least 500 bar, at least 550 bar, at least 600 bar, at least 650 bar, at least 700 bar, at least 750 bar, at least 800 bar, at least 850 bar, at least 900 bar, at least 950 bar, at least 1000 bar etc.) In some embodiments, methods of the present invention allow for a pressure differential of at least 50 bar (e.g., at least 100 bar, at least 150 bar, at least 200 bar, at least 250 bar, at least 300 bar, at least 350 bar, at least 400 bar, at least 450 bar, at least 500 bar, at least 550 bar, at least 600 bar, at least 650 bar, at least 700 bar, at least 750 bar, at least 800 bar, at least 850 bar, at least 900 bar, at least 950 bar, at least 1000 bar etc.) with a radiant thermal gradient of at most 1 K (e.g., at most 0.9 K, at most 0.8 K, at most 0.7 K, at most 0.6 K, at most 0.5 K, at most 0.4 K, at most 0.3 K, at most 0.2 K, at most 0.1 K, at most 0.09 K, at most 0.08 K, at most 0.07 K, at most 0.06 K, at most 0.05 K, at most 0.04 K, at most 0.02 K, at most 0.01 K, at most 0.005 K, at most 0.001 K, etc). For example, a method described herein may be characterized by a radial thermal gradient of at most 0.1 K at a pressure differential of at least 100 bar. Methods described herein may be of particular use in improving the performance of a diabatic chromatography method characterized by a radial thermal gradient of at least 0.01 K (e.g., at least 0.02 K, at least 0.03 K, at least 0.04 K, at least 0.05 K, at least 0.06 K, at least 0.07 K, at least 0.08 K, at least 0.09 K, at least 0.1 K, etc.).

### Oligonucleotide Analytes

The methods disclosed herein may be used for the separation of analytes based on their elongation under high-flow conditions. In preferred embodiments, methods of the present disclosure relate to the separation of nucleic acids, including DNA, RNA, or mixtures thereof.

In some embodiments, methods of the present disclosure relate to methods of separating a plurality of oligonucleotides. In some embodiments, the plurality of oligonucleotides includes a plurality of single-stranded DNA (ssDNA). In some embodiments, the plurality of oligonucleotides includes a plurality of double-stranded DNA (dsDNA). In some embodiments, the plurality of oligonucleotides includes a plurality of RNA. In some embodiments, the plurality of oligonucleotides includes a plurality of double-stranded RNA (dsRNA). In some embodiments, the plurality of oligonucleotides include a plurality of a first oligonucleotide (e.g., DNA (e.g., ssDNA or dsDNA) or RNA (e.g., ssRNA or dsRNA)) and a plurality of a second oligonucleotide (e.g., DNA (e.g., ssDNA or dsDNA) or RNA (e.g., ssRNA or dsRNA)).

In some embodiments, a method described herein may be used to separate a plurality of DNA and a plurality of RNA. In some embodiments, a method described herein may be used to separate a plurality of ssDNA and a plurality of dsRNA. In some embodiments, a method described herein may be used to separate a plurality of a first dsDNA and a plurality of a second dsDNA. In some embodiments, a method described herein may be used to separate a plurality of dsDNA and a plurality of ssDNA. In some embodiments, a method described herein may be used to separate a plurality of dsRNA and a plurality of ssRNA.

In embodiments wherein the plurality of oligonucleotides include dsDNA, the dsDNA may include linear dsDNA, circular dsDNA, looped dsDNA, or supercoiled dsDNA. In some embodiments, the plurality of oligonucleotides includes only one conformation of DNA (e.g., only linear dsDNA, only circular dsDNA, only looped dsDNA, only supercoiled dsDNA). In some embodiments, the plurality of oligonucleotides includes a mixture of two or more conformations of DNA.

In embodiments wherein the plurality of oligonucleotides include ssRNA, the ssRNA may include linear ssRNA, circular ssRNA, looped ssRNA, or circular ssRNA. In some embodiments, the plurality of oligonucleotides includes only one conformation of RNA (e.g., only linear RNA, only circular ssRNA, only looped ssRNA, only supercoiled ssRNA). In some embodiments, the plurality of oligonucleotides includes a mixture of two or more conformations of RNA.

In some embodiments, the plurality of oligonucleotides comprises an oligonucleotide of from about 1 to 1000000 base pairs (e.g., from about 1000 to about 100000 base pairs). For example, nucleic acids to be separated by the present methods may be of from about 1000 base pairs to about 2000 base pairs, from about 2000 base pairs to about 3000 base pairs, from about 3000 base pairs to about 4000 base pairs, from about 4000 base pairs to about 5000 base pairs, from about 5000 base pairs to about 7500 base pairs, from about 7500 base pairs to about 10000 base pairs, from about 10000 base pairs to about 12500 base pairs, from about 12500 base pairs to about 15000 base pairs, from about 15000 base pairs to about 17500 base pairs, from about 17500 base pairs to about 20000 base pairs, from about 20000 base pairs to about 25000 base pairs, from about 25000 base pairs to about 30000 base pairs, from about 30000 base pairs to about 35000, form about 35000 to about 40000 base pairs, from about 40000 base pairs to about 45000 base pairs, from about 45000 base pairs to about 50000 base pairs, from about 50000 base pairs to about 55000 base pairs, from about 55000 base pairs to about 60000 base pairs, from about 60000 base pairs to about 65000 base pairs, from about 65000 base pairs to about 70000 base pairs, from about 70000 base pairs to about 75000 base pairs, from about 75000 base pairs to about 80000, from about 80000 base pairs to about 85000 base pairs, from about 85000 base pairs to about 90000 base pairs, from about 90000 base pairs to about 95000 base pairs, or from about 95000 base pairs to about 100000 base pairs.

### Chromatography Systems

The methods disclosed herein may be used with any high-performance liquid chromatography (HPLC) or ultra-high performance liquid chromatography (UHPLC) system. In some embodiments, an HPLC or UHPLC chromatography system may include a liquid chromatography column, a jacket surrounding the liquid chromatography column, and a vacuum chamber formed in an area between the liquid chromatography column and the jacket. In some embodiments, the chromatography systems may further include a detector in fluid communication with the liquid chromatography system, configured to detect one or more analytes (e.g., oligonucleotides, e.g., DNA or RNA) in the eluent.

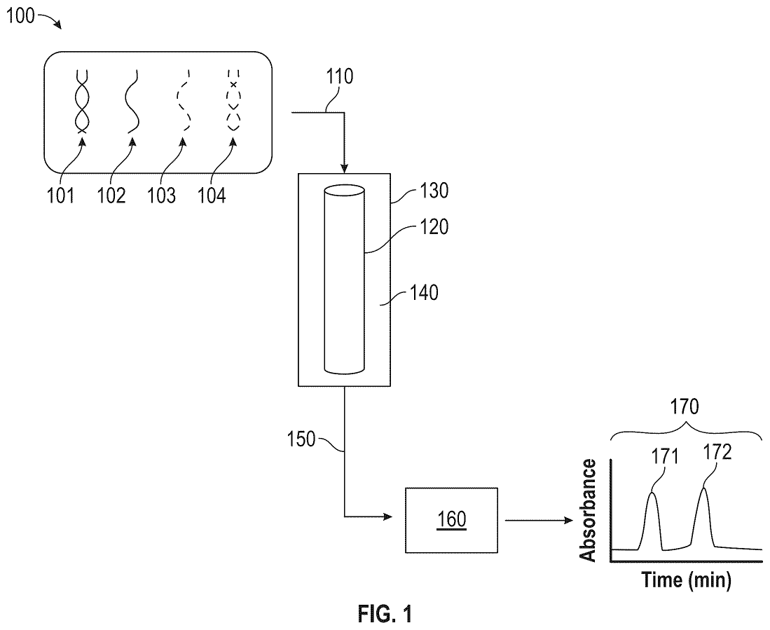

FIG. 1 shows an exemplary chromatography system of the present invention. The chromatography system includes inlet end 110 of chromatography column 120. Chromatography column 120 is surrounded by a vacuum chamber 140, defined as the space between the walls of the chromatography column and a vacuum jacket 130. Surrounding chromatography column 120 with vacuum jacket 140, and putting the area between under vacuum to form vacuum chamber 130 puts chromatography column 120 under adiabatic conditions. The sample of oligonucleotides 100 flows from chromatography column 120 through the outlet end 150 to detector 160. Detector 160 then produces chromatogram 170. The methods of the present invention feature the ability to produce a plurality of distinct peaks (e.g., distinct peak 171 and distinct peak 172).

The liquid chromatography column (e.g., chromatography column 120) includes a column body with an inlet region (e.g., inlet region 110) and an outlet region (e.g., inlet region 110), and contains a chromatography material within the column body. The chromatography material may be packed.

The choice of column and particle, particularly with respect to column length and particle size, is in part dependent on the size of the nucleic acid molecules to be separated and detected as would be appreciated and readily understood by one of ordinary skill in the art. In some embodiments, the column and particle are chosen such that the chromatography experiment produces a desired zone retention factor (k₁). In some embodiments, the desired zone retention factor is less than 0.35. In some embodiments, the desired zone retention factor is from about −0.25 to 0.35. In some embodiments, the desired zone-retention factor is about 0. In some embodiments, the column and particle are chosen such that the elution of a desired analyte from the column can be completed in a desired time. In some embodiments, the desired time is from about 0.6 minutes to about 10 minutes. In some embodiments, the desired time is from about 1 minute to about 3 minutes.

A chromatography system disclosed herein may further include one or temperature sensors, each temperature sensor configured to sense a temperature along a different portion of a length of the chromatography column (e.g., a temperature sensor configured to sense the temperature of the inlet region of the liquid chromatography column, a temperature sensor configured to sense the temperature of the outlet region of the liquid chromatography column, or a temperature sensor configured to sense the temperature of a middle region between the inlet region and the outlet region). In some embodiments, the chromatography system further includes a plurality of temperature sensors (e.g., at least 2 temperature sensors, at least 3 temperature sensors, at least 4 temperature sensors, at least 5 temperature sensors, at least 10 temperature sensors, at least 15 temperature sensors, at least 20 temperature sensors, at least 30 temperature sensors, etc.). In some embodiments, the plurality of temperature sensors include at least an inlet temperature sensor configured to determine the temperature of the inlet region and an outlet temperature sensor configured to determine the temperature of the outlet region. In some embodiments, the chromatography system further comprises at least one processor operative to execute the computer-executable code to cause the apparatus to determine the temperature of the liquid chromatography column from the plurality of temperature sensors, and individual control at least one of the temperature sensors based on the inlet temperature or the outlet temperature. In some embodiments, the processor may communicate with one or more openings in the vacuum chamber. In some embodiments, one of the one or more openings is in fluid communication with a vacuum pump. In some embodiments, one of the one or more openings is in fluid communication with a source of inert gas. By modulating communication with a vacuum pump and a source of inert gas, the processor may control the thermal conductivity of the vacuum chamber.

In some embodiments, a chromatography system of the present disclosure is characterized by an effective thermal conductivity (i.e., the transfer of heat throughout a system composed of a multitude of components). The effective thermal conductivity depends in part on the thermal conductivity of each individual component of the chromatography system. For example, the effective thermal conductivity of a chromatography system may depend on the thermal conductivity of the chromatographic material and the thermal conductivity of the mobile phase. In some embodiments, the effective thermal conductivity of the chromatography system is at most 2 W/m·K (e.g., at most 1.4 W/m·K). In some embodiments, the effective thermal conductivity of the chromatography system is from about 0.2 W/m·K to about 1.4 W/m·K.

### Chromatography Material

Chromatography columns described herein include a chromatography material within the column body of the liquid chromatography column. In one embodiment, the chromatography material is a plurality of particles (e.g., a plurality of porous particles, a plurality of non-porous particles, or a plurality of superficially porous particles). Alternatively, the chromatography material may be a monolith.

In some embodiments, the chromatography material is a plurality of particles. The plurality of particles may be porous or non-porous. In some embodiments, the porous or non-porous particles are inorganic silica particles, organic particles, or inorganic/organic hybrid particles. Examples of particles suitable for use include, but are not limited to, ethylene bridged hybrid particles (BEH; comprising tetraethoxysilane (TEOS) and bis(triethoxysilyl)ethane as described in U.S. Pat. Nos. 6,686,035, and 7,250,214, incorporated herein by reference. Additional particle compositions include, for example, hybrid inorganic/organic particles such as those described in U.S. Pat. Nos. 11,291,974 and 9,145,481; the particles of which are incorporated herein by reference. Additional particles may include polystyrene, divinylbenzene, polyacrylamides, and polymethylacrylates with varying degrees of crosslinking.

In some embodiments, the plurality of particles may include a diameter of from about 1 μm to about 10.5 μm (e.g., from about 1 μm to about 2 μm, from about 2 μm to about 3 μm, from about 3 μm to about 4 μm, from about 4 μm to about 5 μm, from about 5 μm to about 6 μm, from about 6 μm to about 7 μm, from about 7 μm to about 8 μm, from about 8 μm to about 9 μm, from about 9 μm to about 10 μm, or from about 10 μm to about 10.5 μm). In some embodiments, the plurality of particles comprise a diameter of from about 1.5 μm to about 1.9 μm. In some embodiments, the plurality of particles include a diameter of from about 2.8 μm to about 3.2 μm. In some embodiments, the plurality of particles include a diameter of from about 4.8 μm to about 5.2 μm. In some embodiments, the plurality of particles include a diameter of from about 9.8 μm to about 10.2 μm. In some embodiments, the plurality of particles include a diameter of about 1.7 μm. In some embodiments, the plurality of particles include a diameter of about 3.0 μm. In some embodiments, the plurality of particles include a diameter of about 5.0 μm. In some embodiments, the plurality of particles include a diameter of about 10 μm.

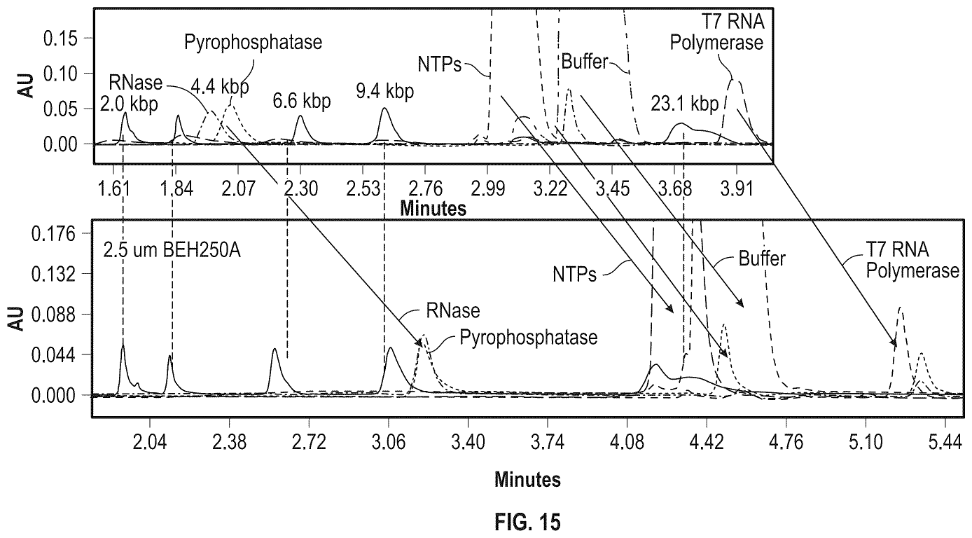

In some embodiments, the particles have an average pore diameter of between 20 Å to 900 Å. In some embodiments, the average specific pore volume of the plurality of particles is from about 0.5 cm³/g to about 1.3 cm³/g. Without wishing to be bound by any particular theory, increasing the pore size shifts the retention of larger molecules relative to the smaller nucleic acid species. Example 9 and FIG. 15 demonstrate the effect of pore size on the retention of nucleic acid species and additional larger components, such as proteins and buffer components.

The plurality of particles may include a charged surface. In some embodiments, the charged surface is a negatively charged surface. The negatively charged surface contributes to electrostatic repulsion of the negatively charged nucleic acid molecules and the particle surface. Without wishing to be bound by any particular theory, distance of the nucleic acid molecules from the particle surface sharpens peak widths and increases separation speed at a constant resolution. In some embodiments, the electrostatic interaction (in preferred embodiments, electrostatic repulsion) is further adjusted by altering the ionic strength of the mobile phase, e.g., by increasing or decreasing the salt content of the mobile phase.

Methods of the present disclosure are particularly suitable for use in combination with thermally insulating chromatography materials (e.g., chromatography materials with a thermal conductivity of at most 2 W/m·K. In some embodiments, the chromatography material may be characterized by a thermal conductivity of at most 2 W/m·K (e.g., at most 1.5 W/m·K, at most 1.0 W/m·K, at most 0.5 W/m·K at most 0.25 W/m·K, or at most 0.1 W/m·K).

### Column Body

The column body of a liquid chromatography column described herein may be characterized by an inner diameter, a length, and a material composition. In some embodiments, the inner diameter of the column body is from about 2.1 mm to about 7.8 mm. In some embodiments, the column body length may be from about 10 mm to about 300 mm. Exemplary column dimensions include, but are not limited to, 2.1×20 mm, 2.1×50 mm, 2.1×100 mm, 2.1×150 mm, 4.6×50 mm, 4.6×100 mm, 4.6×150 mm, and 4.6×300 mm. Exemplary column body materials include stainless steel, polyetheretherketone (PEEK) lined steel, titanium, or a stainless alloy.

In some embodiments, an interior surface of the column body is treated to reduce non-specific binding and enhance overall efficiency of the chromatographic system. In particular, an alkylsilyl coating or other high performance surface is provided to limit or reduce non-specific binding of a sample with walls or interior surfaces of a column body. Without wishing to be bound by theory, it is believed that an alkylsilyl coating covering metal surfaces prevents or minimizes contact between fluids passing through the column body and the interior surfaces of the column. Typically, the alkylsilyl coating is applied to metal surfaces defining what is known as a wetted path of the column. A metal wetted path includes all surfaces formed from metal that are exposed to fluids during operation of the chromatographic column. The metal wetted path includes not only column body walls but also metal frits disposed within the column.

In general, the alkylsilyl coating is applied through a vapor deposition technique. Precursors are charged into a reactor in which the part to be coated is located. Vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating comprises a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. In some embodiments, the high-performance surface is a C2-PEG. A C2-PEG coating may be prepared as described in U.S. Publication No. 2022/0118443. For example, an organosilane precursor, such as bis(trichlorosilyl)ethane or bis(trimethoxysilyl)ethane, may first be vapor deposited on a metal surface, including an interior surface of the column or a metal frit of the column. Following vapor deposition of the organosilane precursor, the coated metal components may be treated with a toluene solution of a polyethylene glycol (PEG), for example 2-[methoxy(polyethyleneoxy)₆₋₉propyl]tris(dimethylaminosilane). The reagent solution may be reacted with the metal components for ~3 days, washed in toluene, washed in isopropanol, and vacuum dried at 70° C., resulting in a C2-PEG coating. Other embodiments of alkylsilyl coatings suitable for use with the present technology are described in U.S. Patent Publication No. 2019/0086371 and U.S. Application Publication No. 2022/0118443 (which are hereby incorporated by reference).

### Vacuum Chamber

Methods of the present invention adiabatically reduce and/or eliminate temperature gradients in high-flow chromatography columns by performing the chromatography experiment under adiabatic conditions. In some embodiments, the adiabatic conditions are achieved using a vacuum chamber (e.g., vacuum chamber 130).

Vacuum chambers of the present invention (e.g., vacuum chamber 130) are formed by placing a vacuum jacket (e.g., vacuum chamber 140) around the exterior of the chromatography column body. The vacuum jacket may be formed from any appropriate material which may withstand reduced pressures, e.g., steel. The vacuum jacket includes an opening in fluid communication with a vacuum pump. The opening may include a means of manipulating the opening such that fluid communication between the vacuum pump and the vacuum chamber may be interrupted, thereby isolating the vacuum chamber.

The vacuum chamber may be characterized by a thermal conductivity of at most 0.2 W/m·K. The thermal conductivity of the vacuum chamber may be controlled by modulating, e.g., the gas within the vacuum chamber or the pressure of the vacuum chamber.

The vacuum chamber may include an inert gas at an appropriate pressure, such that the thermal conductivity of the vacuum chamber is at most 0.2 W. In some embodiments, the inert gas includes carbon dioxide, argon, sulfur hexafluoride, krypton, xenon, or a combination thereof. In some embodiments, the total gas pressure within the vacuum chamber is at most 100 mbar (e.g., at most 50 mbar, at most 10 mbar, at most 5 mbar, at most 1 mbar, at most 0.5 mbar, at most 0.1 mbar, at most 0.05 mbar, at most 0.01 mbar, at most 0.005 mbar, at most 0.001 mbar, etc.).

In some embodiments, the inert gas is characterized by a mean free path within the vacuum chamber. In some embodiments, the mean free path of the inert gas is at least 100 times larger than the width of the opening in the vacuum chamber. In some embodiments, the mean free path of the inert gas is at least 1000 times larger than the width of the opening in the vacuum chamber.

### Detector

According to embodiments of the present disclosure, the columns are connected in fluidic series to a detector (e.g., detector 160). In one aspect, the detector is an ultraviolet (UV) or a tunable ultraviolet (TUV) detector. In some embodiments, the detector is a multi-angle light scattering (MALS) detector. In some embodiments, the UV or TUV detector measures at between 210 nm to 300 nm. In preferred embodiments, the UV or TUV detector measures at between 230 to 260 nm, or more preferably at 230 nm and 260 nm. Said wavelengths are known in the art to detect nucleic acid molecules, including DNA and RNA. Additional detectors, such as fluorescence spectroscopy or mass spectrometry detectors can be utilized in conjunction with the disclosed methods. The detectors can be used alone or in tandem and can be further adjusted to detect molecule(s) of interest. For example, and not by way of limitation, a fluorescence detector may be utilized if the sample comprises a fluorescent molecule of interest.

### Methods of Performing Chromatography Experiments

Described herein are methods of performing a chromatography experiment including the steps of: (a) loading a sample (e.g., a sample including one or more analytes e.g., a first oligonucleotide and a second oligonucleotide) onto the chromatography system; (b) eluting the sample by contacting the liquid chromatography column with a mobile phase; and (c) detecting one or more components of the sample (e.g., detecting the first oligonucleotide and the second oligonucleotide).

The methods described herein feature the resolution of one or more distinct peaks during the detecting step. In some embodiments, each distinct peak results a single type of oligonucleotide. For example, in a method of separating DNA from RNA, the DNA may be resolved as a single distinct peak, and the RNA may be resolved as a single distinct peak. In some embodiments, each distinct peak results from a single size of oligonucleotide. For example, in a method of separating DNA including a first size and DNA including a second size, the first size DNA may be resolved as a single distinct peak and the second size DNA may be resolved as a single distinct peak. In some embodiments, each distinct peak corresponds to a single size of a single oligonucleotide. For example, in a method of separating DNA including a first DNA size and a second DNA size and RNA including a first RNA size and a second RNA size; the first size DNA may be resolved as a single distinct peak, the second size DNA may be resolved as a single distinct peak, the first RNA size may be resolved as a single distinct peak, and the second RNA size may be resolved as a single distinct peak.

Methods of the present invention may be characterized by providing a pressure differential of at least 50 bar (e.g., at least 100 bar, at least 150 bar, at least 200 bar, at least 250 bar, at least 300 bar, at least 350 bar, at least 400 bar, at least 450 bar, at least 500 bar, at least 550 bar, at least 600 bar, at least 650 bar, at least 700 bar, at least 750 bar, at least 800 bar, at least 850 bar, at least 900 bar, at least 950 bar, at least 1000 bar etc.), and creating a radial temperature gradient of at most 1 K (e.g., at most 0.9 K, at most 0.8 K, at most 0.7 K, at most 0.6 K, at most 0.5 K, at most 0.4 K, at most 0.3 K, at most 0.2 K, at most 0.1 K, at most 0.09 K, at most 0.08 K, at most 0.07 K, at most 0.06 K, at most 0.05 K, at most 0.04 K, at most 0.02 K, at most 0.01 K, at most 0.005 K, at most 0.001 K, etc) within the column. For example, a method described herein may be characterized by applying a pressure differential of at least 100 bar, and creating a thermal gradient of at most 0.1 K within the column.

In some embodiments, the chromatography experiment is performed at a temperature of from about 25° C. to about 50° C. In some embodiments, the chromatography experiment is performed at a temperature of from about 25° to about 30° C., from about 30° C. to about 35° C., from about 35° C. to about 40° C., from about 40° C. to about 45° C., or from about 45° C. to about 50° C. In general, increases in temperature reduce shear rates. In some embodiments, the temperature at which the chromatography experiment is performed is altered to optimize the performance of the column. In general, methods of the present disclosure reduce the thermal gradient (e.g., an axial thermal gradient or a radial thermal gradient) in an adiabatic chromatography column compared to a similar chromatography column under diabatic conditions across all applicable temperatures.

In some embodiments, a method described herein is characterized by a flow rate (i.e., the rate at which the mobile phase moves through the chromatography column). Without wishing to be bound by theory, it should be apparent to those of skill in the art that the flow rate for a chromatography experiment is in part dependent on the pressure the chromatography experiment is performed at, and the void space of the chromatography material.

In some embodiments, the flow rate of the chromatography experiment is from about 0.1 mL/min to about 15 mL/min. For example, the flow rate may be from about 0.1 mL/min to about 0.5 mL/min, from about 0.5 mL/min to about 1.0 mL/min, from about 1.0 mL/min to about 1.5 mL/min, from about 1.5 mL/min to about 2.0 mL/min, from about 2.0 mL/min to about 2.5 mL/min, from about 2.5 mL/min to about 3.0 mL/min, from about 3.0 mL/min to about 3.5 mL/min, from about 3.5 mL/min to about 4.0 mL/min, from about 4.0 mL/min to about 4.5 mL/min, from about 4.5 mL/min to about 5.0 mL/min, from about 5.0 mL/min to about 5.5 mL/min, from about 5.5 mL/min to about 6.0 mL/min, from about 6.0 mL/min to about 6.5 mL/min, from about 6.5 mL/min to about 7.0 mL/min, from about 7.0 mL/min to about 7.5 mL/min, from about 7.5 mL/min to about 8.0 mL/min, from about 8.0 mL/min to about 8.5 mL/min, from about 8.5 mL/min to about 9.0 mL/min, from about 9.0 mL/min to about 9.5 mL/min, from about 9.5 mL/min to about 10 mL/min, from about 10 mL/min to about 10.5 mL/min, from about 10.5 mL/min to about 11 mL/min, from about 11.5 mL/min to about 12 mL/min, from about 12 mL/min to about 12.5 mL/min, from about 12.5 mL/min to about 13 mL/min, from about 13 mL/min to about 13.5 mL/min, from about 13.5 mL/min to about 14 mL/min, from about 14 mL/min to about 14.5 mL/min, or from about 14.5 to about 15 mL/min. In general, increased flow rates increases shear rates.

A method described herein may be further characterized by the selection chromatography conditions employed during the elution step. These conditions may be optimized to achieve a desired chromatography parameter for a given analyte; e.g., a desired zone-retention factor of an analyte, a desired elution time of analyte, a minimum efficiency of the separation, a USP plate height of the chromatography column, or a maximum full-width-at-half-maximum of a peak corresponding to a specific analyte in a chromatogram resulting from the chromatography experiment.

In one aspect disclosed herein is a method of separating a sample comprising DNA and RNA. The method comprises loading a sample comprising DNA and RNA onto a chromatographic column comprising a plurality of porous or non-porous particles, eluting the DNA and RNA from the column, and detecting the DNA and RNA in the eluent. In some embodiments, the DNA is dsDNA and/or ssDNA. In some embodiments, the RNA is dsRNA or ssRNA. In some embodiments, the DNA and RNA are eluted from the column in between about 1-10 minutes, more preferably 1-3 minutes. In some embodiments, the DNA and RNA are eluted with a zone retention factor of less than 0.3. In some embodiments, the eluting is performed with an efficiency of greater than 40,000. Example 4 describes methods of separating a sample comprising DNA and RNA according to some embodiments of the technology.

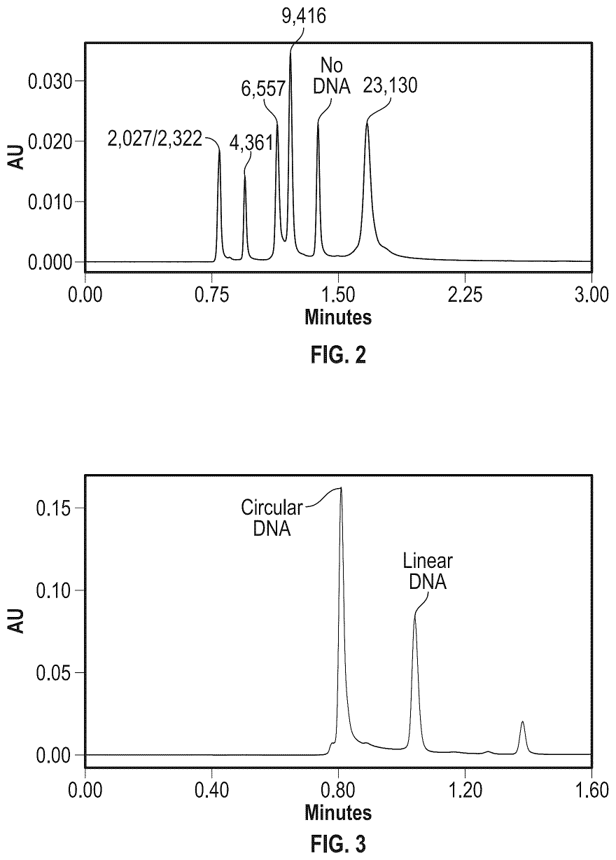

In another aspect, disclosed herein is a method of separating a sample comprising DNA. The method comprises loading a sample comprising DNA onto a chromatographic column comprising a plurality of porous or non-porous particles, eluting the DNA from the column, and detecting the DNA in the eluent. In some embodiments, the DNA is dsDNA and/or ssDNA. In some embodiments, the DNA is eluted from the column in between about 1-10 minutes, more preferably 1-3 minutes. In some embodiments, the DNA is eluted with a zone retention factor of less than 0.3. In some embodiments, the eluting is performed with an efficiency of greater than 40,000. Examples 1-2 and FIGS. 2-3 describe a method of separating a sample comprising DNA according to some embodiments of the present technology.

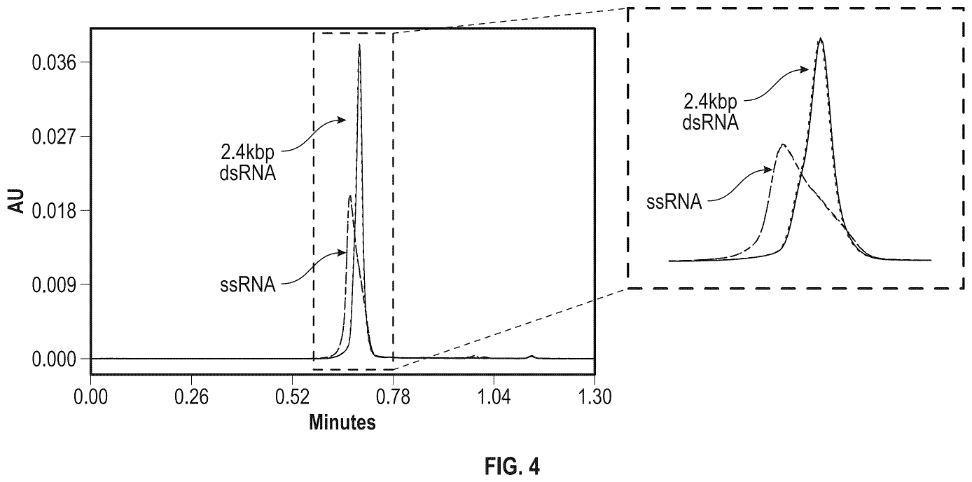

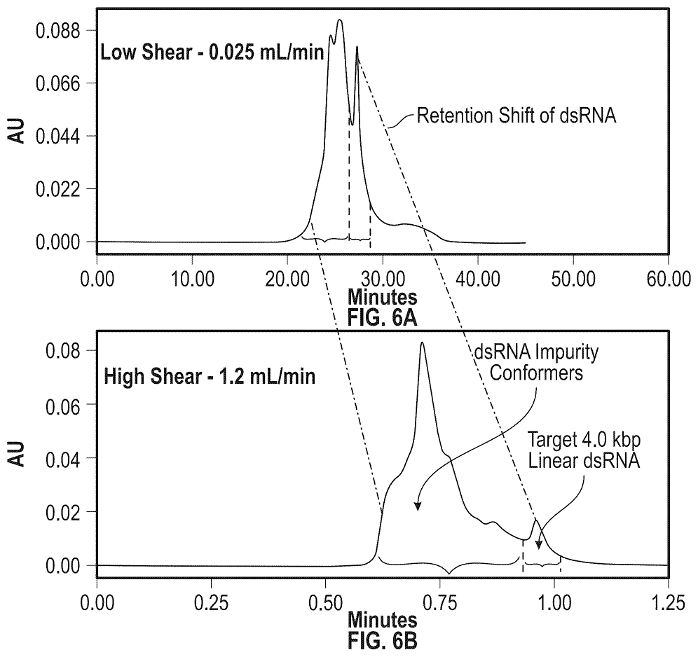

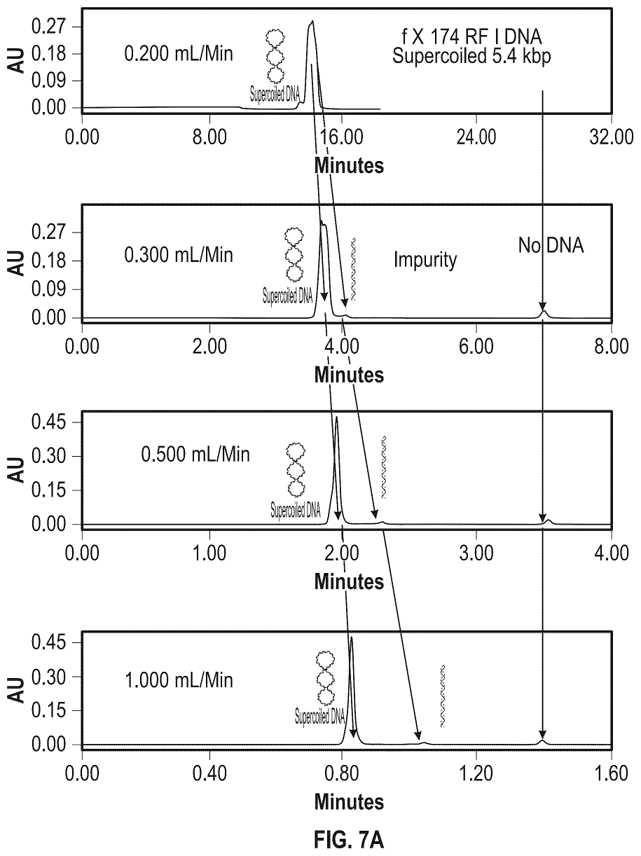

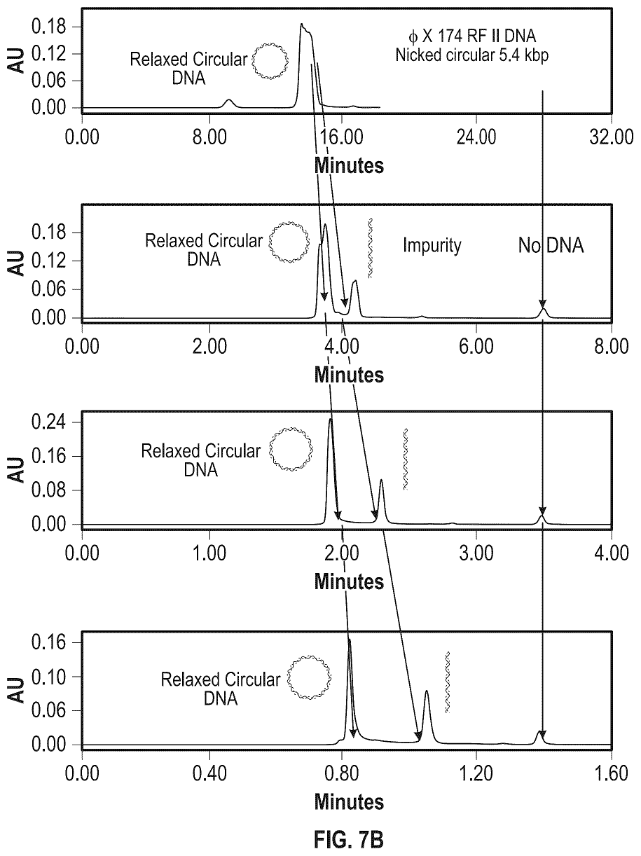

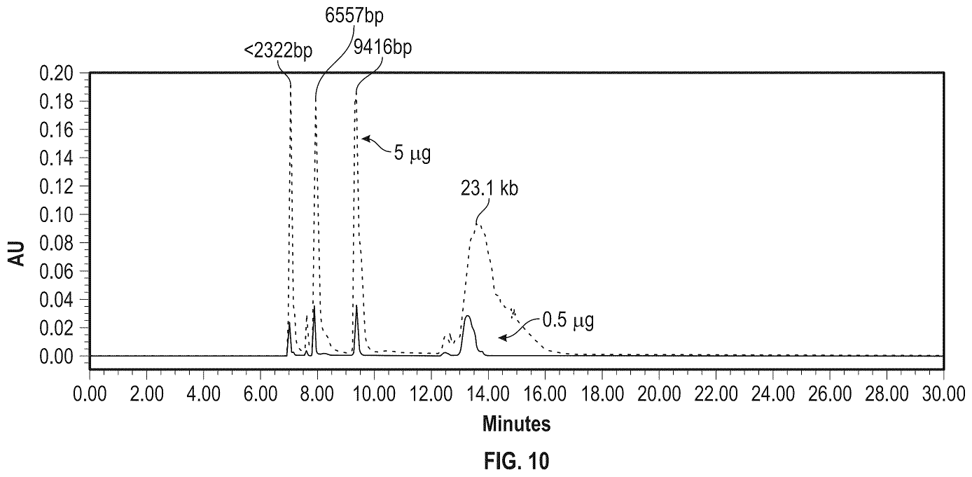

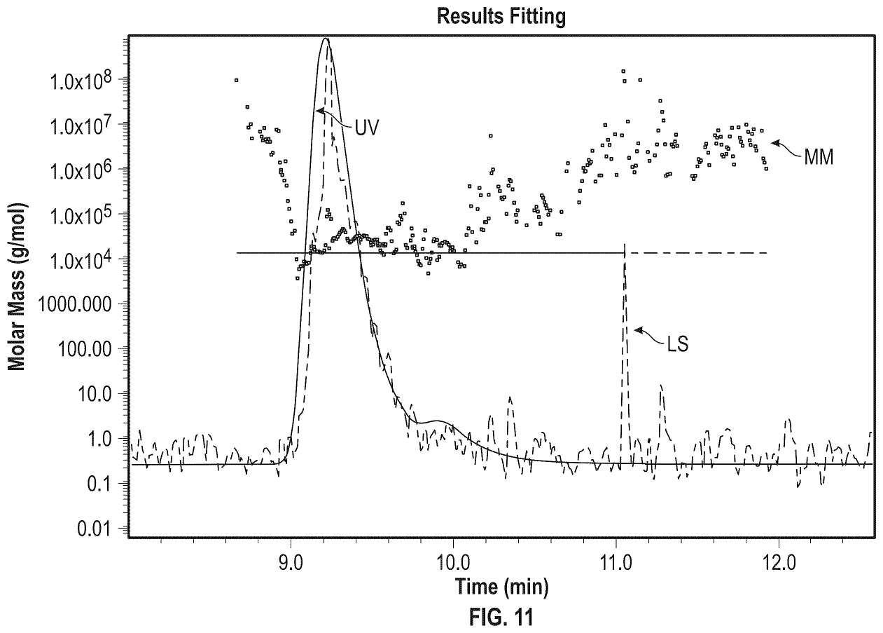

In another aspect, disclosed herein is a method of separating a sample comprising RNA. The method comprises loading a sample comprising RNA onto a chromatographic column comprising a plurality of porous or non-porous particles, eluting the RNA from the column, and detecting the RNA in the eluent. In some embodiments, the RNA is dsRNA and/or ssRNA. In some embodiments, the DNA is eluted from the column in between about 1-10 minutes, more preferably 1-3 minutes. In some embodiments, the DNA is eluted with a zone retention factor of less than 0.3. In some embodiments, the eluting is performed with an efficiency of greater than 40,000. Example 3 and FIG. 4 describe a method of separating a sample comprising RNA according to some embodiments of the present technology. Example 6 and FIGS. 6A-B describe a method of separating linear dsRNA from other dsRNA impurity conformers present in a sample. Example 7 and FIG. 7A-7B describe a method of separating supercoiled DNA or circular DNA from linear DNA impurities. Example 8 and FIGS. 10-11, 12A-12D, and 13-14 describe a method of separating DNA fragments and detecting said fragments with multi-angle light scattering (MALS) and/or dynamic light scattering (DLS).

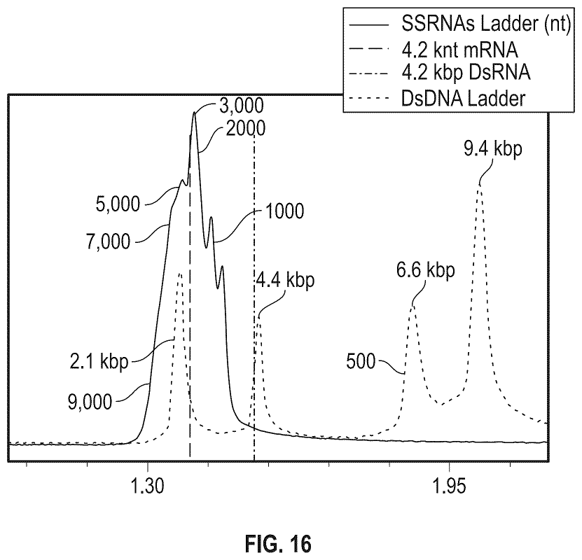

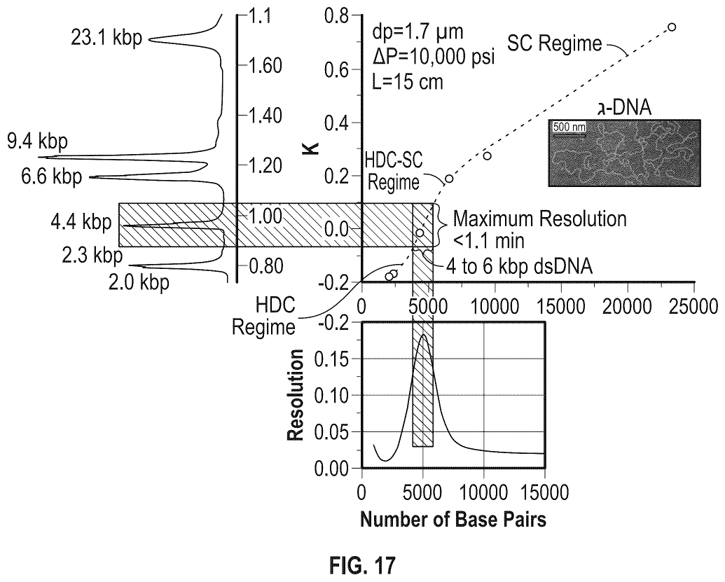

For example, but not by way of limitation, the methods described herein may be used to separate double-stranded RNA (dsRNA) from single-stranded RNA (ssRNA) present in a mixture. As shown in FIG. 16, a sample comprising a 4.2 kb dsRNA and 4.2 kb ssRNA would be resolved using the methods described herein.

In some embodiments of the above methods, the eluent is collected for downstream processing. Examples of downstream processing include, but are not limited to, sequencing of the nucleic acid molecules or cloning of the nucleic acid molecules using methods known in the art.

### Mobile Phase

Methods described herein include eluting the sample from the liquid chromatography column by contacting the liquid chromatography column with a mobile phase. The components of a mobile phase (e.g., the solvent, the buffer, the ionic strength, etc.) are incredibly important factors to consider when performing a chromatography method. In conventional column chromatography, mobile phases typically include a solvent mixture of water and an organic solvent (e.g., acetonitrile, methanol, ethanol, isopropanol, tetrahydrofuran, dimethylsulfoxide, etc.) in order to weaken the electrostatic interactions between the chromatography material and the analyte which otherwise disrupt the movement of the analyte through the chromatography column. Because these organic solvents are typically characterized by lower thermal conductivities than aqueous solvents, thereby decreasing the effective thermal conductivity of chromatography system. To prevent poor thermal conductivity from impacting the results of experiments, typical high-flow chromatography experiments try to reduce the amount of organic solvent present in the mobile phase.

The methods of the present disclosure thermally insulate the chromatography column within a vacuum jacket, thereby improving the ability for the generated heat to dissipate more evenly throughout the column, and reducing thermal gradients. As such, the present methods are particularly suitable to high-flow column chromatography using a low thermal conductivity mobile phase (e.g., at most 1 W/m·k, at most 0.9 W/m·k, at most 0.8 W/m·k, at most 0.7 W/m·k, at most 0.6 W/m·k, at most 0.5 W/m·k, at most 0.4 W/m·k, at most 0.3 W/m·k, at most 0.2 W/m·k, at most 0.1 W/m·k, etc.). In some embodiments, the low thermal conductivity mobile phase includes one or more organic solvents (e.g., acetonitrile, methanol, ethanol, isopropanol, tetrahydrofuran, dimethylsulfoxide, etc.).

In some embodiments, the viscosity of the mobile phase is adjusted. Methods of altering the viscosity of the mobile phase are known in the art, and include, for example, the inclusion of a sugar (e.g., sucrose). In general, increased viscosity increases shear rates. A number of mobile phase buffers are suitable for use with the disclosed methods as would be understood by one of ordinary skill in the art. In some embodiments, the buffer is phosphate-buffered saline. In some embodiments, the phosphate-buffered saline is at 1× concentration, at 2× concentration, at 0.1× concentration, at 0.0× concentration, or any value between 0.0× and 2× concentration.

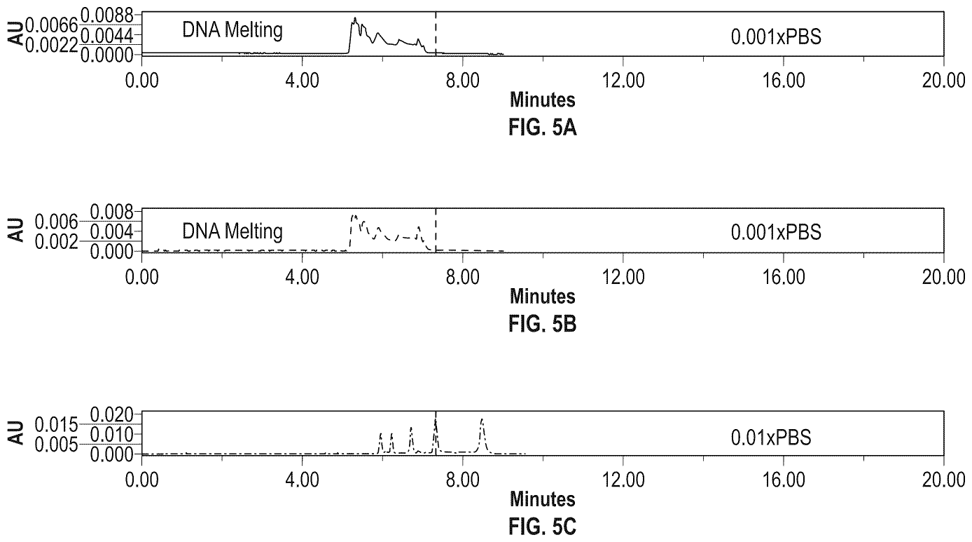

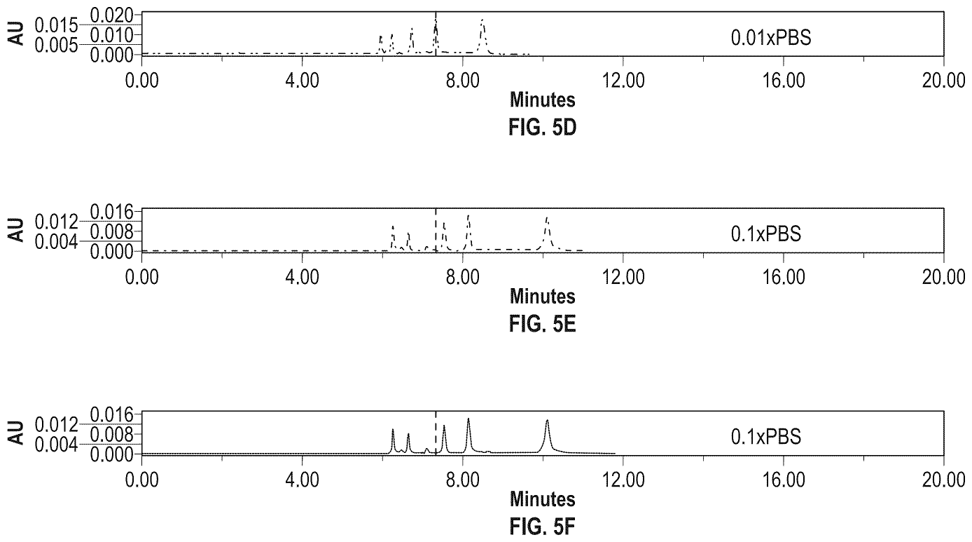

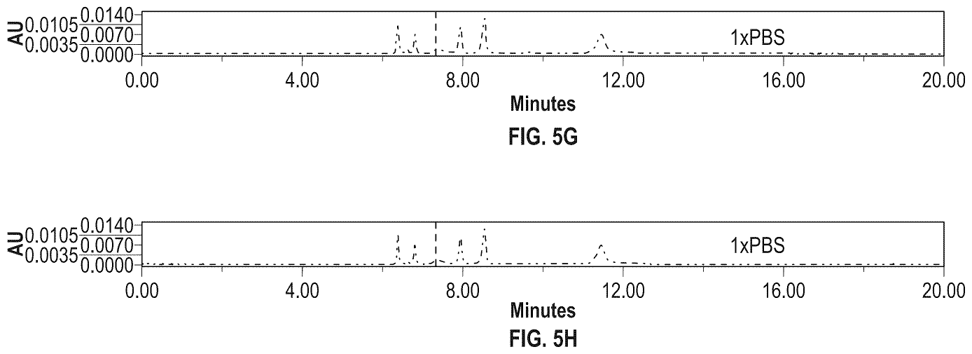

In some embodiments, the elution time and peak resolution of one or more peaks may be modified by adjusting, e.g., the ionic strength of the mobile phase. Example 5 and FIGS. 5A-H demonstrate the impact of ionic strength on elution time and peak resolution (i.e., changes to the k₁ value of said peaks) when separating nucleic acids, such as linear, double-stranded DNA. Examples of negatively charged surfaces include silanols, surface modifications with a silane comprising a pendant group that has a negative charge (e.g., COO⁻ or SO₃⁻), the grafting of a negatively charged group onto a polymer particle, or a chemical modification of a polymer particle that results in the surface display of a negatively charged group as would be understood by one of ordinary skill in the art.

### Methods of Characterizing Analyte Retention

Methods of the present invention are capable of separating one or more analytes within a sample from one another during a chromatography experiment. Broadly, each of the one or more analytes interacts uniquely with the liquid chromatography column (e.g., due to chemical or steric differences between the analytes). This results in each analyte being differently retained by the chromatography column. Analyte retention may be measured herein using, e.g., the zone retention factor (k₁) of the analyte or the elution time for the analyte.

### Zone-Retention Factor (k₁)

The zone-retention factor (k₁) of an analyte refers to the ratio between the retention of an analyte (e.g., a nucleic acid molecule) on a column and the void retention volume (i.e., the volume of elution solvent retained by the column). In other words, k₁ represents the relative retention of an analyte compared to the elution solvent.

The calculation of k₁ depends on the interaction between the analyte and the chromatographic material. For example, when the analyte size is greater than the pore size (e.g., an oligonucleotide with a size of less than 250 Å used in combination with a particle comprising pores of 250 Å or less; or an oligonucleotide of any size in combination with a non-porous particle) zone retention factor may be defined using Equation 2:

$${k}_{1} = {k}_{1 , τ = 0} + ( 1 - {e}^{- τ / {τ}_{C}} ) ( {k}_{1 , MAX} - {k}_{1 , τ = 0} ) ; \qquad Equation 2$$

wherein k_{1,τ=0} refers to the retention of a molecule, e.g., a nucleic acid, in the absence of elongation (e.g., under low flow rates);
wherein τ_{C} refers to the characteristic pressure stress of a molecule, e.g., a nucleic acid;
wherein k_{1,MAX} refers to the maximum stretching of a molecule, e.g., a nucleic acid; and r refers to the shear stress pressure variable, and may be defined using Equation 3

$$τ = 〈 η 〉 \frac{u}{{d}_{p}} ; \qquad Equation 3$$

wherein η is the dynamic viscosity of the mobile phase;
u is the average linear interstitial velocity of the mobile phase; and
dₚ is the particle diameter.

When the analyte size is less than the pore size (e.g., an oligonucleotide with a size of about 250 Å used in combination with a particle comprising pores of 250 Å or more), then the particle porosity more strongly impacts the traversal of the analyte through the chromatography system. To correct for this, a mixed retention mechanism shifting the value of the zone retention factor as calculated by the “universal retention factor formula” is employed.

The universal retention factor formula is defined by Equation 4:

$${k}_{1} = ( 1 - ∅ ) {k}_{1 , HDC} + ∅ {k}_{1 , SC} + {k}_{1 , SE C} ; \qquad Equation 4$$

wherein k_{1,SEC} is the zone retention factor under size exclusion chromatography conditions (i.e., relative retention is dominated by steric factors);
k_{1,HDC} is the zone retention factor under hydrodynamic chromatography conditions (i.e., relative retention is dominated by chemical factors);
k_{1,SC} is the zone retention factor under slalom chromatography conditions (i.e., retention under high flow conditions); and
Ø is the fraction of the retention factor supported by a strict slalom chromatography conditions. 1−Ø, then, is the fraction of the retention factor supported by strict hydrodynamic chromatography conditions.
   k_{1,SEC} may be defined through Equation 5:

$${k}_{1 , S EC} = \frac{1 - {ε}_{e}}{{ε}_{e}} {ε}_{p} ( 1 - 4.4 ( \frac{{R}_{g}}{{D}_{meso}} ) + 4.98 {( \frac{{R}_{g}}{{D}_{meso}} )}^{2} ; \qquad Equation 5$$

wherein R_{g} is the gyration radius of the oligonucleotide defined by Equation 6:

$${R}_{g} = 0.116 * L {c}^{0.57} ; \qquad Equation 6$$

Dₘₑₛₒ refers to the size of the mesopores on the particle;
wherein εₑ is the inter-particle void fraction of the column; and
εₚ is the internal porosity of the particle.
   k_{1,HDC} may be defined through Equation 7:

$${k}_{1 , HDC} = \frac{1}{1 + 2 ( \frac{{D}_{rep}}{{D}_{pore}} ) - 2.698 ( {( \frac{{D}_{rep}}{{D}_{pore}} )}^{2} )} - 1 ; \qquad Equation 7$$

wherein Dₚₒᵣₑ refers to the average distance between particles, and
Dᵣₑₚ is the hydrodynamic diameter of extended oligonucleotide. Dᵣₑₚ may be defined by Equation 8:

$${D}_{rep} = 4 \sqrt{\frac{{( \frac{\sqrt{π}}{2} {R}_{g} )}^{3}}{3 〈 {L}_{ext} 〉}} ; \qquad Equation 8$$

wherein R_{g} is the gyration radius defined through Equation 6; and
<Lₑₓₜ> is the average extension length of the oligonucleotide under shear flow conditions.
   k_{1,SC} may be defined by Equation 9:

$${k}_{1 , SC} = ( 1 - {e}^{- \frac{τ}{{τ}_{C}}} ) * α * {L}_{contour} ; \qquad Equation 9$$

wherein α is a proportionality empirical constant independent of the contour length of the oligonucleotide (for DNA, α=0.1 mm⁻¹);
τ_{C} is a characteristic shear stress of the oligonucleotide chain (t_{C}=0.25 Pa for dsDNA);
τ is the shear stress pressure variable defined using Equation 2; and
L_{contour} is the fully extended length of the oligonucleotide.

In the context of the Universal Retention Factor Formula, k_{1,SC} defines the variable <Lₑₓₜ>. When the oligonucleotide is DNA, α=0.1 mm⁻¹ and τ_{C}=0.25 Pa. Similar values may be used to approximate the k₁ value for RNA oligonucleotides.

Without wishing to be bound by any particular theory, it is understood that multiple chromatographic principles may influence retention of an analyte (e.g., a DNA or RNA molecule) based on the size and relaxation time (ms) of the analyte. For example, under increased flow rate (i.e., high shear rates), analytes with longer relaxation times will have increased retention on a column. In this context, k₁ may be defined using the universal retention factor formula described above.

In some embodiments, the zone retention factor of an analyte is less than 0.35. In some embodiments, the zone retention factor of an analyte is from about −0.25 to 0.35. In some embodiments, the zone-retention factor is about 0.

### Elution Time

Elution time, as used herein, refers to the time between injecting an analyte (e.g., at the inlet region of the chromatography column) and the time for the entirety of the analyte to elute from the column (e.g., at the outlet region of the chromatography column). Without wishing to be bound by any particular theory, it will be understood that elution time is dependent on multiple chromatographic principles, including zone retention factor of the analyte and the length of the column. Accordingly, characterizing the performance of a chromatography system by its elution time may be indicative of the zone retention factor the analyte (e.g., at a particular column length).

In some embodiments, the elution time of an analyte may be from about 0.6 minutes to about 10 minutes. In some embodiments, the elution time of an analyte may be from about 1 minute to about 3 minutes. In some embodiments, the elution time of n analyte is less than 10 minutes.

### Methods of Characterizing the Performance of a Chromatography Experiment

The present disclosure relates to the heretofore unanticipated benefits observed when performing high-flow column chromatography under adiabatic conditions. These benefits may be described herein through a variety of parameters. For example, the benefits provided by the present invention may be expressed in terms of a selectivity factor (a) between two analytes, the chromatography experiment efficiency (N), or the USP plate height of the chromatography experiment. Alternatively, or additionally, the benefit may be expressed through changes observed in a chromatogram resulting from the chromatography experiment. For example, the benefit may be expressed in terms of the full-width-at-half-maximum (FWHM) of one or more distinct peaks observed in a chromatogram. In some embodiments, the performance of a method disclosed herein may be given under specific column conditions (e.g., at a specific flow rate, or column length; or under column conditions wherein the analyte has a specific zone retention factor or elution time).

### Selectivity Factor (α)

The selectivity factor (α) of a chromatography experiment is used to express the relative separation between two analytes (e.g., a first analyte and a second analyte) in a sample. The selectivity factor can be determined using Equation 10:

$$α = \frac{( {t}_{R 2} - {t}_{0} )}{( {t}_{R 1} - {t}_{0} )} ; \qquad Equation 10$$

wherein is the void elution time (i.e., the elution time of the mobile phase through the chromatography material solvent);
t_{R1} is the elution time of the first analyte; and
t_{R2} is the elution time of the second analyte.

In some embodiments, the selectivity factor between two analytes in a chromatography experiment described herein is at least 1 (e.g., at least 1.1, at least 1.2, at least 1.3, at least 1.4, at least 1.5, at least 1.6, at least 1.7, at least 1.8, at least 1.9, at least 2, etc.)

### Efficiency (N)

As used herein, the term “efficiency” or “N” with respect to chromatography analysis refers to a measurement of peak dispersion. N may be defined using Equation 11:

$$N = L / {d}_{p} ( \frac{1}{3} - \frac{3}{4} {k}_{1} + \frac{2}{3} {k}_{1}^{2} - \frac{1}{4} {k}_{1}^{3} ) ; \qquad Equation 11$$

wherein L is the column length;
wherein dₚ is the particle diameter; and
wherein k₁ is the zone retention factor.

In some embodiments, the efficiency of a chromatography experiment may be at least 1000 (e.g., at least 2000, at least 3000, at least 4000, at least 5000, at least 10000, at least 20000, at least 30000, at least 40000, at least 50000, etc.).

### Resolution Factor (R_{S})

The resolution factor (R_{S}) of a chromatography experiment is a measure of how well separated two signals resulting from two separate analytes are from one another in a chromatography experiment. The resolution between two peaks (i.e., two distinct peaks) in a chromatography experiment may be expressed through a resolution factor. R_{S} may be defined through Equation 12.

$${R}_{S} = \frac{{k}_{1}}{{k}_{1} + 1} \times \frac{α - 1}{α} \times \frac{\sqrt{N}}{4} \qquad Equation 12$$

Notably, Rₛ combines a term for the retention factor

$$( \frac{{k}_{1}}{{k}_{1} + 1} ) ,$$

a term for the selectivity of the chromatography experiment

$$( \frac{α - 1}{α} ) ,$$

and a term for the efficiency of the chromatography experiment

$$( \frac{\sqrt{N}}{4} ) .$$

Accordingly, Rₛ may be used as a cumulative metric summarizing the impact of retention factor, selectivity, and efficiency of a chromatography experiment on the resolution of two peaks. In general, a larger R_{S} value indicates better resolution between two peaks. It is generally desirable for R_{S} to be large enough that two peaks are well resolved, but not so large as to unnecessarily increase the time a chromatography experiment must be performed for. In some embodiments, methods of the present disclosure are performed under conditions such that R_{S} is from about 1 to about 2. In preferred embodiments, R_{S} is about 1.5.

### USP Plate Height (H)

In some embodiments, the performance of a chromatography system is defined in terms of the USP Plate Height (H) of the column. USP Plate Height may be calculated using Equation 13.

$$H = \frac{{P}_{N}}{L} ; \qquad Equation 13$$

wherein L is the length of the chromatography column; and
P_{N} is the theoretical plate number of the chromatography experiment, which may be calculated using Equation 14:

$${P}_{N} = 5.54 {( \frac{{t}_{R}}{{W}_{h}} )}^{2} ; \qquad Equation 14$$

wherein t_{R} is the retention time of the relevant peak in the chromatography experiment; and
Wₕ is the full-width-at-half-maximum of the peak relevant peak in the chromatography experiment.

In some embodiments, a chromatography system including a vacuum jacket (as described herein) may be characterized by an increase in USP plate height compared to a chromatography system not including a vacuum jacket. In some embodiments, the increase in USP plate height is at least 10%. In some embodiments, the increase in USP plate height is at least 20%. In some embodiments, the increase in USP plate height is at least 30%.

### Full-Width-Half-Max of Distinct Peaks

In some embodiments, the performance of a chromatography system is defined in terms of the full-width-at-half-maximum (also referred to herein as “full-width-half-max”, or “FWHM”). The FWHM of a given peak is determined by calculating the width of the peak at a signal intensity (e.g., a UV signal intensity) of half of the maximum intensity reached by said peak. In general, for a chromatography experiment examining a signal height across a timescale, narrower peaks (i.e., peaks with a lower FWHM) are desirable, as they indicate more of an analyte is eluting at a single time, thereby increasing the relative purity of a resulting sample. Moreover, in some separations, two distinct analytes elute at similar times, resulting in overlapping signals. These overlapping signals may appear as only one peak in the resulting chromatogram. By improving the separation performance (thereby narrowing the signals resulting from each analyte), the chromatography system is better able to distinguish between the two distinct analytes, improving separation.

In some embodiments, a chromatography system including a vacuum jacket (as described herein) may be characterized by a decrease in FWHM compared to a chromatography system not including a vacuum jacket. In some embodiments, the decrease in FWHM is at least 10%. In some embodiments, the decrease in FWHM is at least 20%. In some embodiments, the decrease in FWHM is at least 30%.

### EXAMPLES

The following Examples are provided to illustrate the invention and are not intended to in any way limit the invention to the embodiments disclosed therein.

### Example 1: Separation of Linear Double-Stranded DNA

Linear double-stranded DNA was separated using the methods disclosed herein. A Lambda DNA (λ-DNA) double-stranded DNA (dsDNA) sample (48,502 base pairs/nucleotides in length) was used (available from Thermo Scientific™). The sample was digested using a restriction enzyme to produce six, linear dsDNA fragments, of approximately 2,027, 2,322, 4,361, 6,557, 9,416, and 23,130 base pairs in length. The sample was loaded onto a 4.6 mm×15 cm column comprising porous 1.7 μm BEH particles having 45 Å pore sizes. The column hardware was coated with a C2-PEG high-performance surface. The samples were flowed through the column with a flow rate of 1 mL/min using 100 mM phosphate-buffered saline (pH 8). As shown in FIG. 2, the method afforded robust separation and baseline resolved peaks of the expected dsDNA fragments (with the 2,027 and 2,322 fragments co-eluting) in under 2.25 minutes. The peak labeled no DNA is due to small molecules present in the sample and entering the mesopores of the 1.7 μm BEH45 particles. The k₁ value for the 2,027 and 2,322 bp peak was −0.16; for the 4,361 bp peak was 0.01; for the 6,557 bp peak was 0.24; for the 9,416 bp peak was 0.31; and for the 23,130 bp peak was 0.8. Increasing the flow rate or particle size could afford a lower k1 value for the 23,130 bp fragment.

### Example 2: Separation of Circular and Linear Double-Stranded DNA

The ability to separate DNA molecules with different topologies using the methods disclosed herein was determined. A nicked, circular double-stranded DNA fX174 RF II, 5,386 sample (5,386 base pairs/nucleotides in length) was used (available from NEB). ~90% of the sample comprises nicked circular DNA, and the remainder is supercoiled or linear.

The sample was loaded onto a 4.6 mm×15 cm column comprising porous 1.7 μm BEH particles having 45 Å pore sizes. The column hardware was coated with a C2-PEG high-performance surface. The samples were flowed through the column with a flow rate of 1 mL/min using 100 mM phosphate-buffered saline (pH 8). As shown in FIG. 3, the method afforded robust selectivity between the circular and linear forms of the DNA sample in under 1.2 minutes. In contrast, separating the same sample using size-exclusion chromatography resulted in poor selectivity between the two forms with long elution times of 12-15 minutes (data not shown). Thus, the methods disclosed herein are capable of separating similar length samples with differing topologies. The k₁ value for this separation was 0.15 for the linear DNA. The unlabeled, third peak did not comprise DNA.

### Example 3: Separation of Single- and Double-Stranded RNA

The ability to separate single-stranded RNA (ssRNA) and double-stranded RNA (dsRNA) using the methods disclosed herein was determined. A KH20 RNA sample, ~2,400 base pairs/nucleotides in length, was used (available from New England Biolabs).

The sample was loaded onto a 4.6 mm×150 mm column comprising porous 1.7 μm BEH particles having 45 Å pore sizes. The column hardware was coated with a C2-PEG high-performance surface. The samples were flowed through the column with a flow rate of 1 mL/min using 100 mM phosphate-buffered saline (pH 8). As shown in FIG. 4, the method could distinguish between ssRNA and dsRNA forms of the RNA sample in under 1 minute (with the dashed box representing a blown-out portion of the chromatograph). As such, the disclosed methods afford rapid detection of ssRNA purity in a sample. The k₁ value for this separation was −0.24 for the 2.4 kb dsRNA sample.

### Example 4: Separation of DNA/RNA by Flow Rate and Particle Size

The relationship between particle size, flow rate, and nucleic acid size was determined. Single-stranded RNA (ssRNA), single-stranded DNA (ssDNA), double-stranded RNA (dsRNA), and double-stranded DNA (dsDNA) of varying lengths were tested, and selectivity factors for the separation of the double-stranded molecules from the single-stranded molecules (α_{ds/ss}) or the separation of the double-stranded DNA from the double-stranded RNA (α_{ds2/ds1}) were determined. The experiments were repeated using particle sizes of 1.7 μm, 3.0 μm, 5.0 μm, and 10 μm, with a range of flow rates using a 4.6 mm inner diameter column. The particles were BEH porous (average pore size of ~125 Å). The column hardware was coated with a C2-PEG high-performance surface.

A summary of the relaxation time (τ_{R}) and the gyration radius (R_{G}) for the DNA/RNA molecules tested is shown in Table 1.

TABLE 1
Summary of Parameters for Tested Nucleic Acids

| | | Nucleic Acid Length | | | | | |
|---|---|---|---|---|---|---|---|
| Nucleic | | (kilonucleotides/kilobasepairs) | | | | | |
| Acid | Parameter | 1 | 5 | 10 | 25 | 50 | 100 |
| SSDNA/ | Relaxation | 0.015 | 0.2 | 0.8 | 4 | 12 | 40 |
| ssRNA | Time (ms) | | | | | | |
| | Gyration | 0.023 | 0.052 | 0.073 | 0.12 | 0.16 | 0.23 |
| | Radius | | | | | | |
| | (μm) | | | | | | |
| dsDNA/ | Relaxation | 0.4 | 6 | 20 | 90 | 300 | 1000 |
| dsRNA | Time | | | | | | |
| | (ms) | | | | | | |
| | Gyration | 0.063 | 0.16 | 0.23 | 0.39 | 0.58 | 0.87 |
| | Radius | | | | | | |
| | (μm) | | | | | | |

The ability to separate the nucleic acid molecules were first tested using a 1.7 μm particles at flow rates 0.1 mL/min, 0.5 mL/min, or 1.0 mL/min. When relevant, selectivity factors were also calculated using hydrodynamic chromatographic principles (HDC; when the j shear rate trends towards zero). Selectivity factors were determined as described above. As shown in Table 2, in general, a particle size of 1.7 μm and a flow rate of >0.5 mL/min afforded good separation as measured by selectivity factors for nucleic acid molecules between 5,000 bp/nt and 25,000 bp/nt, including both between double-stranded species and between single- and double-stranded species.

TABLE 2
Specificity Factors Using a 1.7 μm Particle

| | Selec- | | Nucleic Acid Length | | | | | |
|---|---|---|---|---|---|---|---|---|
| Flow | tivity | | (kilonucleotides/kilobasepairs) | | | | | |
| Rate | Factor | Method | 1 | 5 | 10 | 25 | 50 | 100 |
| 0.1 | α_{ds/ss} | HDC | 0.89 | — | — | — | — | — |
| mL/min | | SC | 1.00 | 1.01 | 1.05 | 1.27 | 1.68 | — |
| | α_{ds2/ds1} | HDC | — | — | — | — | — | — |
| | | SC | — | 1.00 | 1.01 | 1.05 | 1.14 | — |
| 0.5 | α_{ds/ss} | HDC | 0.89 | — | — | — | — | — |
| mL/min | | SC | 1.00 | 1.14 | 1.36 | 2.04 | — | — |
| | α_{ds2/ds1} | HDC | — | — | — | — | — | — |
| | | SC | — | 1.14 | 1.19 | 1.51 | — | — |
| 1.0 | α_{ds/ss} | HDC | 0.89 | — | — | — | — | — |
| mL/min | | SC | 1.02 | 1.21 | 1.47 | 2.24 | — | — |
| | α_{ds2/ds1} | HDC | — | — | — | — | — | — |
| | | SC | — | 1.19 | 1.22 | 1.56 | — | — |

The ability to separate the nucleic acid molecules was then tested using 3.0 μm particles at flow rates 0.25 mL/min, 0.75 mL/min, or 1.5 mL/min. When relevant, selectivity factors were also calculated using hydrodynamic chromatography (HDC; a method that differs from slalom chromatography due to the low shear rates). Selectivity factors were determined as described above. As shown in Table 3, in general, a particle size of 3 μm and a flow rate of >0.25 mL/min afforded good separation as measured by selectivity factors for nucleic acid molecules between 5,000 bp/nt and 50,000 bp/nt, including both between double-stranded species and between single- and double-stranded species. A flow rate of 0.25 mL/min was capable of separating nucleic acid species up to 100 kbp/knt in length.

TABLE 3
Specificity Factors Using a 3.0 μm Particle

| | | | Nucleic Acid Length | | | | | |
|---|---|---|---|---|---|---|---|---|
| Flow | Selectivity | | (kilonucleotides/kilobasepairs) | | | | | |
| Rate | Factor | Method | 1 | 5 | 10 | 25 | 50 | 100 |
| 0.25 | α_{ds/ss} | HDC | 0.92 | 0.86 | 0.86 | — | — | — |
| mL/min | | SC | 1.00 | 1.01 | 1.06 | 1.23 | 1.54 | 2.15 |
| | α_{ds2/ds1} | HDC | — | 0.88 | 0.96 | — | — | — |
| | | SC | — | 1.01 | 1.04 | 1.17 | 1.26 | 1.42 |
| 0.75 | α_{ds/ss} | HDC | 0.92 | 0.86 | 0.86 | — | — | — |
| mL/min | | SC | 1.00 | 1.07 | 1.19 | 1.54 | 2.12 | — |
| | α_{ds2/ds1} | HDC | — | 0.88 | 0.96 | — | — | — |
| | | SC | — | 1.07 | 1.11 | 1.31 | 1.41 | — |
| 1.5 | α_{ds/ss} | HDC | 0.92 | 0.86 | 0.86 | — | — | — |
| mL/min | | SC | 1.01 | 1.11 | 1.25 | 1.68 | 2.29 | — |
| | α_{ds2/ds1} | HDC | — | 0.88 | 0.96 | — | — | — |
| | | SC | — | 1.10 | 1.13 | 1.36 | 1.45 | — |

The ability to separate the nucleic acid molecules was then tested using 5.0 μm particles at flow rates 1.0 mL/min, 2.5 mL/min, or 5.0 mL/min. When relevant, selectivity factors were also calculated using hydrodynamic chromatography (HDC; a method that differs from slalom chromatography due to the low shear rates). Selectivity factors were determined as described above. As shown in Table 4, in general, a particle size of 5 μm and a flow rate of >1.0 mL/min afforded good separation as measured by selectivity factors for nucleic acid molecules between 5,000 bp/nt and 50,000 bp/nt, including both between double-stranded species and between single- and double-stranded species. A flow rate of 1.0 mL/min was capable of separating nucleic acid species up to 100 kbp/knt in length.

TABLE 4
Specificity Factors Using a 5.0 μm Particle

| | Selec- | | Nucleic Acid Length | | | | | |
|---|---|---|---|---|---|---|---|---|
| Flow | tivity | Meth- | (kilonucleotides/kilobasepairs) | | | | | |
| Rate | Factor | od | 1 | 5 | 10 | 25 | 50 | 100 |
| 1.0 | α_{ds/ss} | HDC | 0.94 | 0.89 | 0.86 | 0.86 | — | — |
| mL/ | | SC | 1.0 | 1.03 | 1.09 | 1.29 | 1.60 | 2.12 |
| min | α_{ds2/ds1} | HDC | — | 0.90 | 0.94 | 0.95 | — | — |
| | | SC | — | 1.03 | 1.06 | 1.18 | 1.27 | 1.43 |
| 2.5 | α_{ds/ss} | HDC | 0.94 | 0.89 | 0.86 | 0.86 | — | — |
| mL/ | | SC | 1.0 | 1.07 | 1.15 | 1.40 | 1.74 | — |
| min | α_{ds2/ds1} | HDC | — | 0.9 | 0.94 | 0.95 | — | — |
| | | SC | — | 1.03 | 1.06 | 1.18 | 1.27 | — |
| 5.0 | α_{ds/ss} | HDC | 0.94 | 0.89 | 0.86 | 0.86 | — | — |
| mL/ | | SC | 1.01 | 1.08 | 1.17 | 1.40 | 1.69 | 2.12 |
| min | α_{ds2/ds1} | HDC | — | 0.90 | 0.94 | 0.95 | — | — |
| | | SC | — | 1.07 | 1.09 | 1.25 | 1.34 | — |

The ability to separate the nucleic acid molecules was lastly tested using 10 μm particles at flow rates 2.5 mL/min, 7.5 mL/min, or 15 mL/min. When relevant, selectivity factors were also calculated using hydrodynamic chromatography (HDC; a method that differs from slalom chromatography due to the low shear rates). Selectivity factors were determined as described above. As shown in Table 5, in general, a particle size of 10 μm and a flow rate of >2.5 mL/min afforded good separation as measured by selectivity factors for nucleic acid molecules between 25,000 bp/nt and 100,000 bp/nt, including both between double-stranded species and between single- and double-stranded species.

TABLE 5
Specificity Factors Using a 10 μm Particle

| | Selec- | | Nucleic Acid Length | | | | | |
|---|---|---|---|---|---|---|---|---|
| Flow | tivity | | (kilonucleotides/kilobasepairs) | | | | | |
| Rate | Factor | Method | 1 | 5 | 10 | 25 | 50 | 100 |
| 2.5 | α_{ds/ss} | HDC | 0.97 | 0.93 | 0.90 | 0.87 | 0.85 | — |
| mL/ | | SC | 1.00 | 1.02 | 1.05 | 1.16 | 1.31 | 1.53 |
| min | α_{ds2/ds1} | HDC | — | 0.94 | 0.96 | 0.93 | 0.95 | — |
| | | SC | — | 1.02 | 1.03 | 1.11 | 1.16 | 1.29 |
| 7.5 | α_{ds/ss} | HDC | 0.97 | 0.93 | 0.90 | 0.87 | 0.85 | — |
| mL/ | | SC | 1.00 | 1.04 | 1.08 | 1.19 | 1.3 | 1.40 |
| min | α_{ds2/ds1} | HDC | — | 0.94 | 0.96 | 0.93 | 0.95 | — |
| | | SC | — | 1.03 | 1.04 | 1.13 | 1.20 | 1.33 |
| 15 | α_{ds/ss} | HDC | 0.97 | 0.93 | 0.90 | 0.87 | 0.85 | — |
| mL/ | | SC | 1.01 | 1.04 | 1.08 | 1.16 | 1.23 | 1.30 |
| min | α_{ds2/ds1} | HDC | — | 0.94 | 0.96 | 0.93 | 0.95 | — |
| | | SC | — | 1.04 | 1.05 | 1.13 | 1.20 | 1.34 |

In sum, the presently disclosed methods are capable of separating nucleic acid molecules and mixtures thereof across a range of nucleotide lengths (ranging from 1,000 to 100,000 base pairs/nucleotides). In general, a larger particle size is able to separate larger nucleic acid molecules, and vice versa (e.g., smaller particle sizes are able to separate smaller nucleic acid molecules. The methods have further demonstrated the ability to separate both single-stranded and double-stranded molecules.

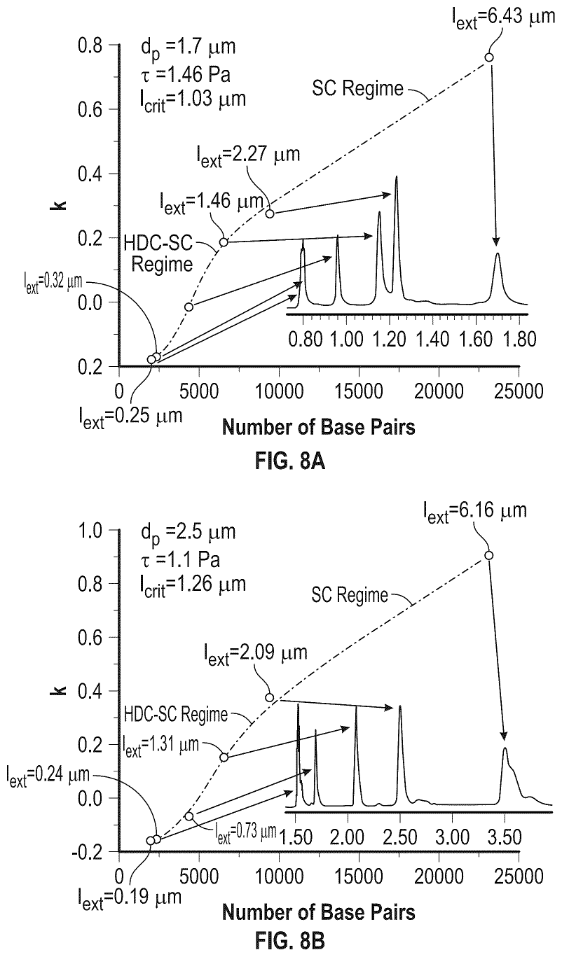

The methods described above further allow for the measurement of dsDNA length using known standards as depicted in FIG. 8A (for 1.7 μm particles) and FIG. 8B (for 2.5 μm particles). FIG. 8A-8B show the correlation between dsDNA length as measured by number of base pairs and the k coefficient.

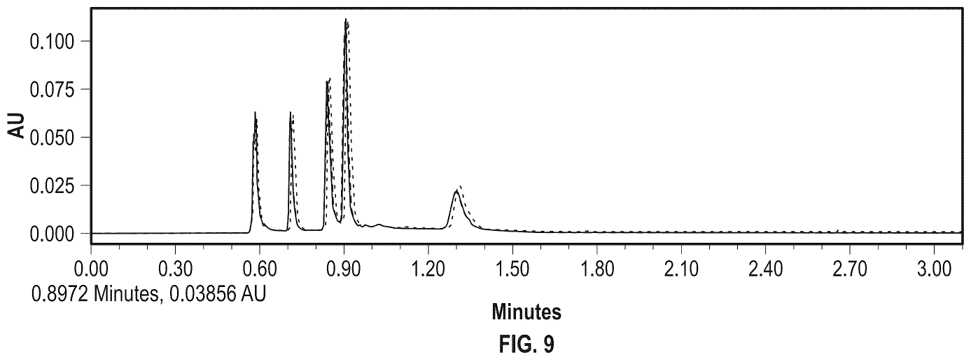

The methods described above further allow for the separation of DNA and RNA using 1.7 μm diethylene bridged hybrid particles (BEH) having a diol-bonded surface and an average pore size of 130 Å as shown in FIG. 9.

### Example 5: Effect of Ionic Strength on the Separation of Double-Stranded DNA

The effect of the ionic strength of the mobile phase on the separation of double-stranded DNA was assessed. A double-stranded DNA (dsDNA) plasmid digest was loaded onto a 4.6 mm×300 mm column comprising 2.6 μm non-porous silica particles and eluted using a mobile phase of 1×PBS (high ionic strength), 0.1×PBS, 0.01×PBS, or 0.001×PBS (low ionic strength). The column hardware was coated with a C2-PEG high-performance surface.

As shown in FIG. 5, decreasing the ionic strength of the mobile phase reduced retention time (e.g., peaks eluted faster) while improving peak resolution and k₁ values of said peaks. At 1×PBS, higher molecular weight peaks eluted past 10 minutes, with k₁ factors above 0.35. With the 2.6 μm non-porous silica particles used in this example, decreasing the ionic strength of the mobile phase from 1×PBS to either 0.1×PBS or, more preferably, 0.01×PBS, resulted in overall improved separation of the dsDNA sample. The results shown for 0.1×PBS and 0.01×PBS illustrate peaks eluted with k₁ factors at or below 0.35. Lowering the ionic strength to 0.001×PBS resulted in DNA melting due to instability of the sample in the mobile phase.

The ionic strength of the mobile phase may further be adjusted to improve peak shape and alter retention time, taking into consideration the sample being tested and the net negative charge of the particles used in the column. For example, adjustment or modification of the surfaces of particles can be controlled to result in a net negative charge of the particle surface, thus affecting the ionic strength conditions of the elution in the column.

### Example 6: Separation of Double-Stranded RNA (dsRNA) Species

A sample of double-stranded RNA (dsRNA) was loaded onto a 4.6 mm×300 mm column comprising diethylene bridged hybrid (BEH) diol particles and eluted using either a low shear flow rate (0.025 mL/min) or high shear flow rate (1.2 mL/min). The column hardware was coated with a C2-PEG high-performance surface. As shown in FIG. 6, increasing the flow rate from 0.025 mL/min to 1.2 mL/min shifted the retention of the dsRNA species, allowing for the separation of impurity conformers and the target, linear dsRNA in less than 1 minute.

### Example 7: Separation of Circular/Supercoiled and Linear DNA Species

A sample comprising either supercoiled DNA (ΦX 174 RF I DNA) or circular DNA (ΦX 174 RF II DNA) were loaded onto a 4.6 mm×150 mm column comprising 1.7 μm ethylene bridged hybrid (BEH) particles having an average pore size of 45 Å. The column hardware was coated with a C2-PEG high-performance surface. Samples were flowed through the column using 100 mM phosphate buffer (pH 8) at 0.2 mL/min, 0.3 mL/min, 0.5 mL/min, or 1 mL/min. As shown in FIG. 7A-7B, increasing flow rate allowed for the separation of linear dsDNA impurities from the supercoiled DNA sample (FIG. 7A) and circular DNA sample (FIG. 7B), respectively.

### Example 8: Detection of Separated Nucleic Acids Using Multi-Angle Light Scattering (MALS)

A sample comprising a lambda DNA-HindIII digest (available from Promega Corporation) was loaded onto a 4.6 mm×300 mm column comprising 2.5 μm diethylene bridged hybrid (BEH) particles having an average pore size of 125 Å. The column hardware was coated with a C2-PEG high-performance surface. Sample was loaded at either a concentration of 5 μg or 0.5 μg and flowed through the column using 1× phosphate buffered saline (PBS) at a 0.2 mL/min flow rate. Eluent was detected using a multi-angle light scattering (MALS) detector. As shown in FIG. 10, the DNA digest could be separated and detected using MALS, particularly at the higher concentration with 0.5 μg representing the lower limit of detection for light scattering.

As a calibrant, a sample comprising a small interfering RNA (siRNA) was loaded onto a 4.6 mm×300 mm column comprising 2.5 μm diethylene bridged hybrid (BEH) particles having an average pore size of 125 Å. The column hardware was coated with a C2-PEG high-performance surface. Sample was loaded at a concentration of 1 μg and flowed through the column using 1×PBS at a 0.2 mL/min flow rate. Eluent was detected using a MALS detector. FIG. 11 provides a chromatograph of the sample detected using MALS. The trace labeled LS and UV represents the light scattering measurement and the ultraviolet measurement, respectively. The black squares represent the molar mass (MM) measurements. The expected monomer mass of the siRNA was 13.2 kDa and the observed, fitted monomer mass was ~12.4 kDa. Thus, siRNA was determined to be a suitable calibrant for MALS detection in conjunction with the provided methods.

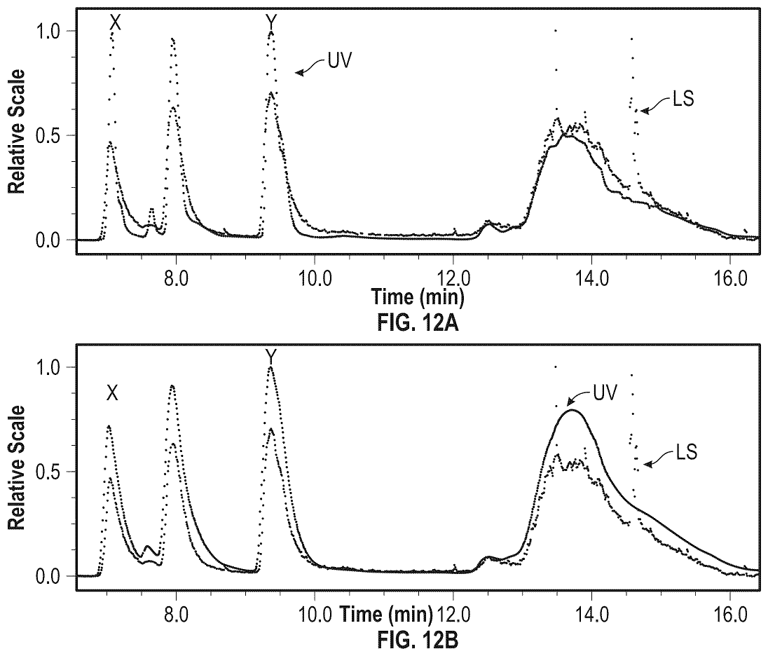

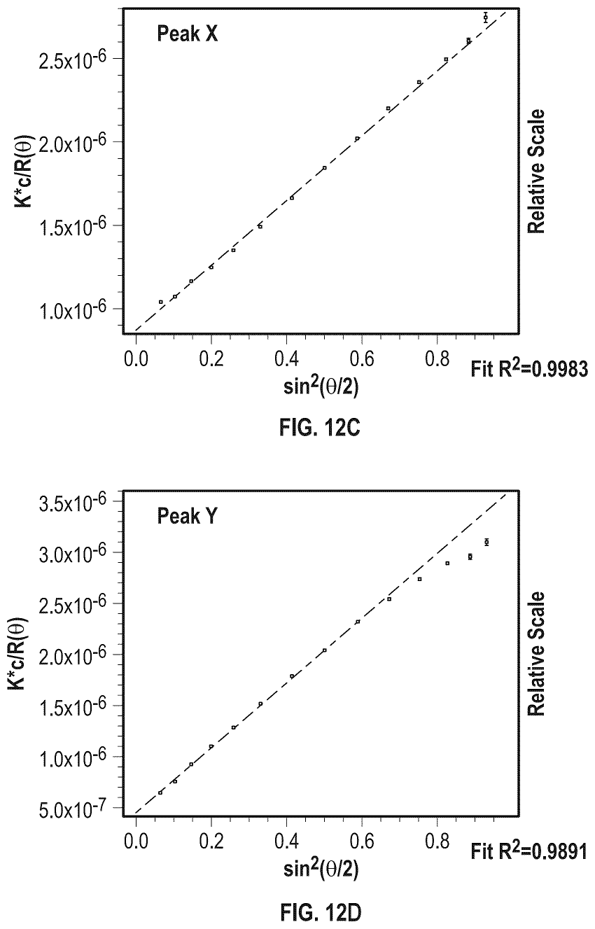

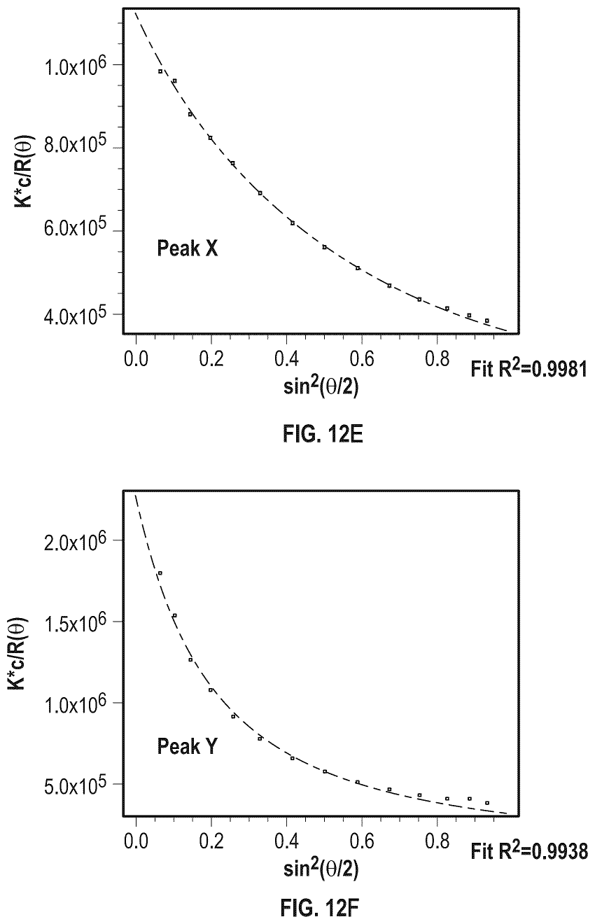

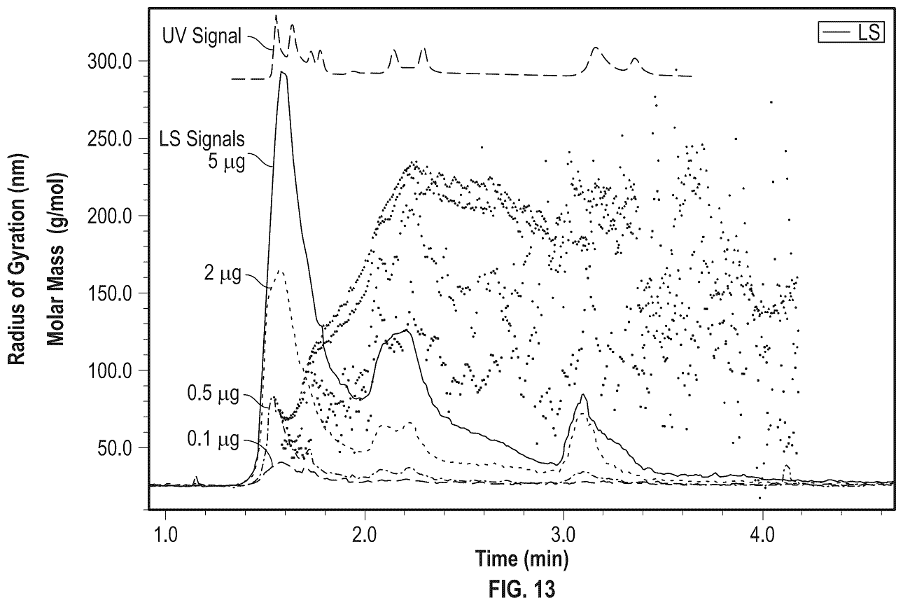

Using siRNA as the calibrant, the DNA-HindIII digest was modeled to determine molar mass. FIG. 12A-12B provide chromatographs of the DNA-HindIII digest before (FIG. 12A) and after (FIG. 12B) broadening of the UV signal. Small peaks (corresponding to lower molecular weight DNA fragments) fit to Zimm formalism as shown in FIG. 12C (Peak X) and FIG. 12 D (Peak Y) and Random Coil as shown in FIG. 12E (Peak X) and FIG. 12F (Peak Y). FIGS. 12C and 12D provide the Zimm plots for peak X and Y as labeled in corresponding FIG. 12A-12B. FIGS. 12E and 12F provide the Random Coil plots for peak X and Y as labeled in corresponding FIG. 12A-12B. Methods of modeling MALS-derived data and determining relevant parameters for said models are known in the art and are described in, for example, U.S. Pat. No. 6,651,009, incorporated herein by reference FIG. 13 demonstrates the light scattering and molar mass measurements for samples detected at 0.1 μg, 0.5 μg, 2 μg, and 5 μg.

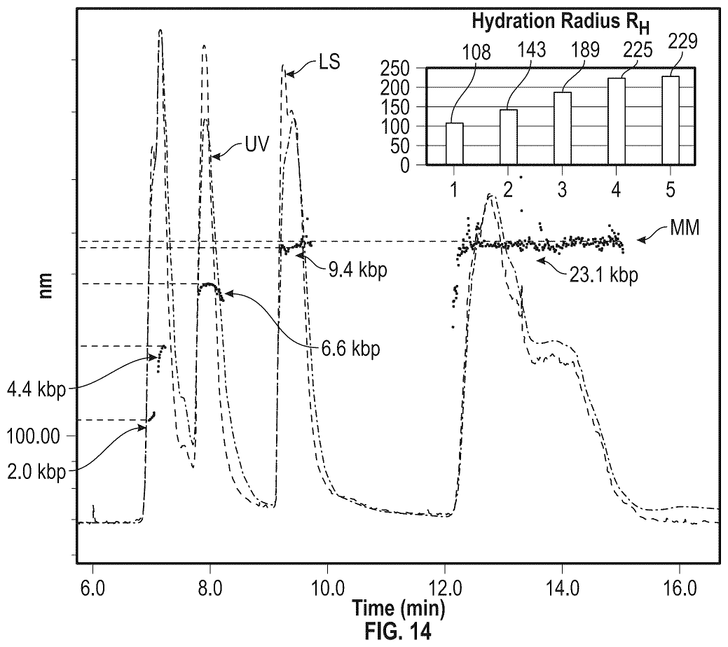

In addition to MALS detection, sample can be detected using dynamic light scattering (DLS) to determine the hydration radius (R_{H}). Sample comprising the lambda DNA digest was loaded onto 4.6 mm×300 mm column comprising 2.5 μm diethylene bridged hybrid (BEH) particles having an average pore size of 125 Å. Sample was flowed through the column with a 0.2 mL/min flow rate and detected using MALS and DLS detectors. FIG. 14 provides a representative chromatograph of the detected nucleic acid species, including both UV and light scattering traces, molar mass measurements (dots), and hydration radius calculations (top right bar graph).

### Example 9: Separation of Nucleic Acids from Large Molecules

The methods of the present technology may be used to separate nucleic acids (such as dsDNA, ssDNA, dsRNA, or ssRNA) from additional large molecules, including biomolecules (proteins such as enzymes) and buffer components. Sample comprising a dsDNA ladder having DNA fragments of 2 kb, 4.4 kb, 6.6 kb, 9.4 kb, and 23.1 kb, RNAse, pyrophosphatase, NTPs, buffer, and T7 RNA polymerase was separated on a column comprising 2.5 μm diethylene bridged hybrid (BEH) particles having either 125 Å or 250 Å pore size. As shown in the top of FIG. 15, the pore size of 125 Å resulted in the co-elution of DNA with additional components present in the sample. In contrast and as shown in the bottom of FIG. 15, when the 250 Å pore size particle is used, DNA fragments ranging from 2 kb to 9.4 kb eluted from the column before the additional components present in the sample. Thus, the 250 Å pore size particle can be used with the provided methods to separate nucleic acids, such as DNA or RNA, from additional components present in a mixture.

### Example 10: Thermal Gradients in Diabatic Chromatography Columns

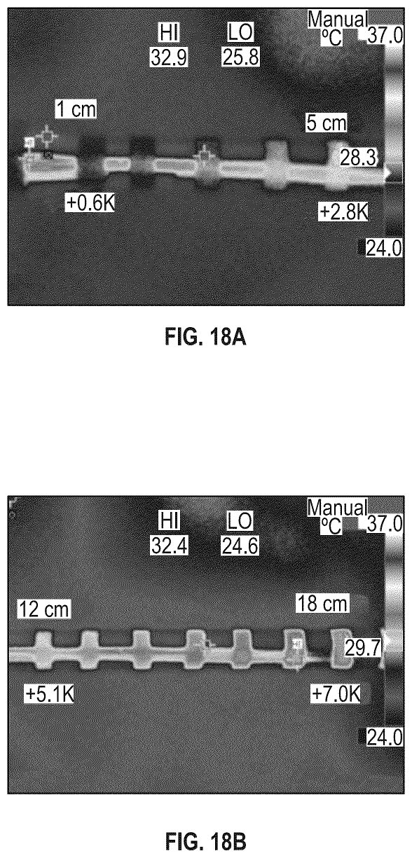

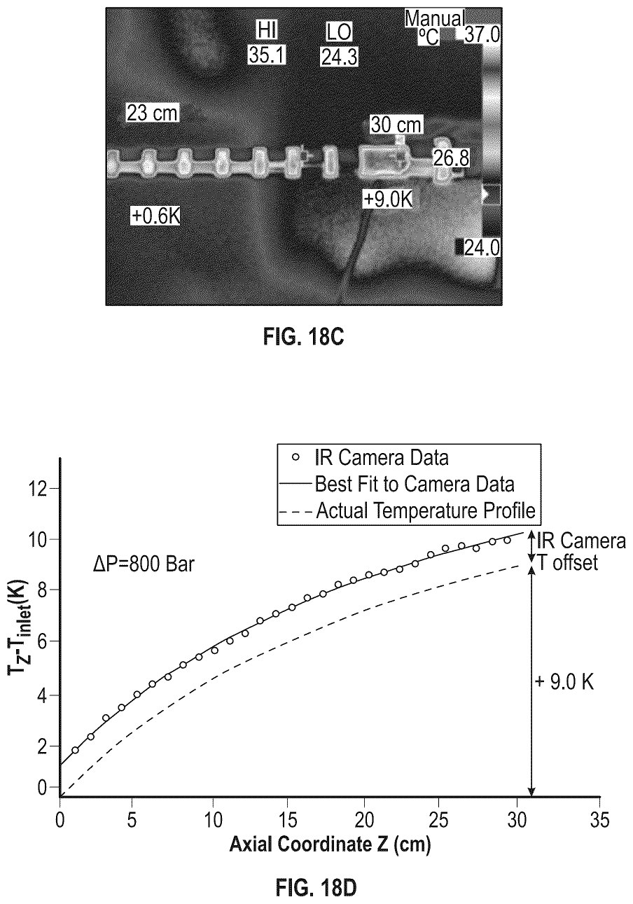

An IR thermometer was attached to a chromatography system including a 4.6×300 mm long column, including 2.8 μm particles, and using a pure water mobile phase. FIGS. 18A-18D shows the relative temperatures of the chromatography column along its axial length at an applied pressure differential from the inlet end of the chromatography column to the outlet end of the chromatography column (ΔP) of 800 bar. FIG. 18A shows the temperature at a representative range near the inlet region of the chromatography column (from 1 cm to 5 cm along the length of the column). FIG. 18B shows the temperature at a representative range near a middle region of the chromatography column (from 12 cm to about 18 cm along the length of the column). FIG. 18C shows the temperature at a representative range near the outlet region of the chromatography column (from about 23 cm to about 30 cm along the length of the column). FIG. 18D plots the measured temperature axial temperature gradient along a distance (z) from the inlet of the chromatography column. The representative, uninsulated, column examined had an axial thermal gradient of about 9 K.

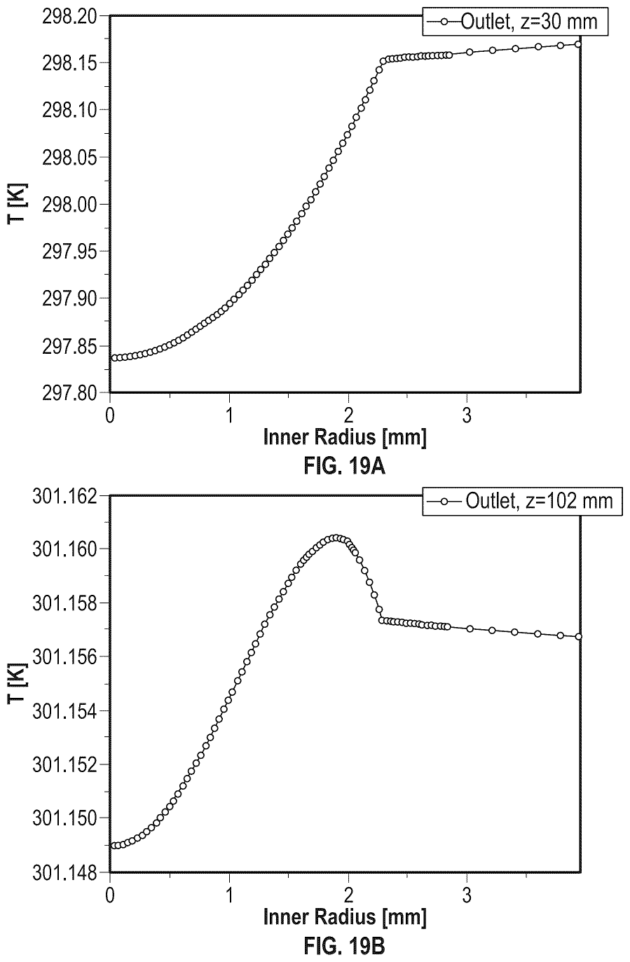

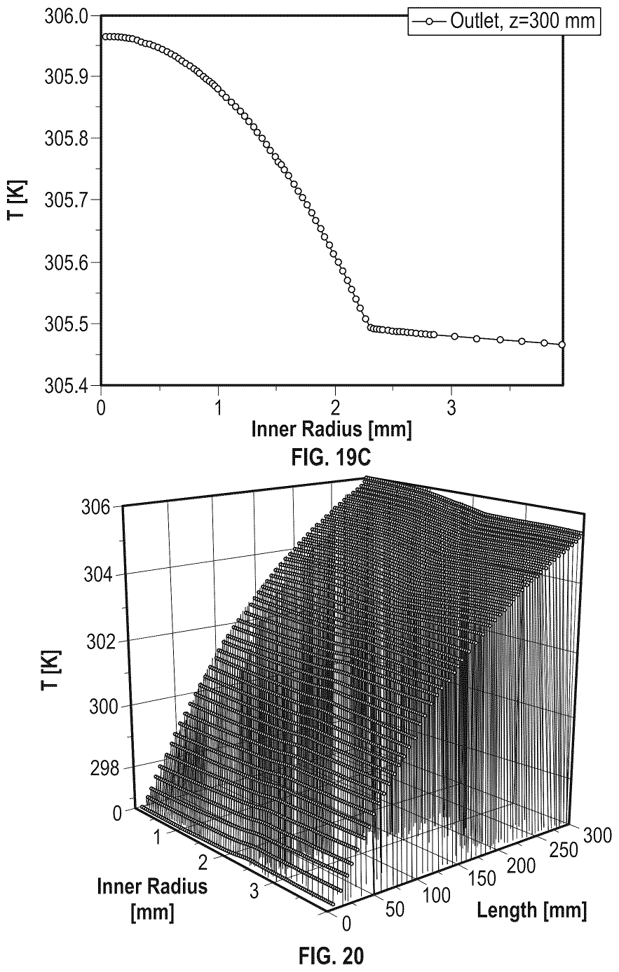

FIGS. 19A-119C plots the radial temperature gradient at three representative points along the length of a representative 2.1×300 mm chromatography column. FIG. 19A shows the radial temperature gradient at approximately 3 cm along the length of the chromatography column. FIG. 19B shows the radial temperature gradient at approximately 10.2 cm along the length of the chromatography column. FIG. 19C shows the radial temperature gradient at approximately 30 cm along the length of the chromatography column. In each case, there is a discontinuity in the graph at approximately 2.1 cm, resulting from the change in material from the chromatography material to the chromatography body. Across the 2.1 cm diameter of the chromatography column, thermal gradients of 0.45 K (at the inlet region), 0.15 (at the middle region), and 0.5 (at the outlet region) are observed.

FIG. 20 shows a 3D plot displaying the change in radial temperature gradient along the length of a 4.6×300 mm chromatography column including 3 μm SEC BEH particles and a pure water mobile phase, at a pressure differential of 800 bar. Across the length of the column, an axial temperature gradient of about 10 K is observed, with the radial temperature varying along the length of the column. The radial temperature gradient was typically from about 0.2 K to about 0.5 K.

### Example 11: Thermal Gradients in Adiabatic Chromatography Columns

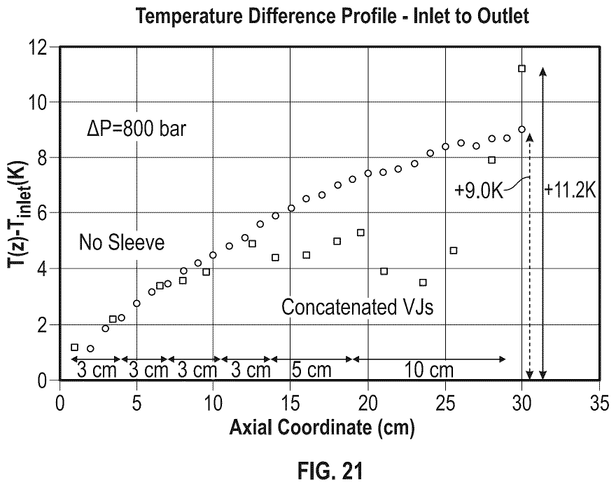

The chromatography system in Example 10 was encased within a series of six concatenated vacuum jackets. A chromatography experiment was then performed under identical conditions (pure water mobile phase and a pressure differential of 800 bar). By performing the experiment under adiabatic conditions, radial temperature gradients were minimized. Notably, the vacuum chamber had a lesser impact on the axial temperature gradient within the column. FIG. 21 shows the relative axial temperature gradient along the length of the chromatography column. Across the length of the column, and particularly within the middle region of the chromatography column, the axial temperature gradient in the adiabatic chromatography column is less than the axial temperature gradient observed in the diabatic chromatography column.

### Example 12: Performance of Chromatography Columns Under Adiabatic Conditions

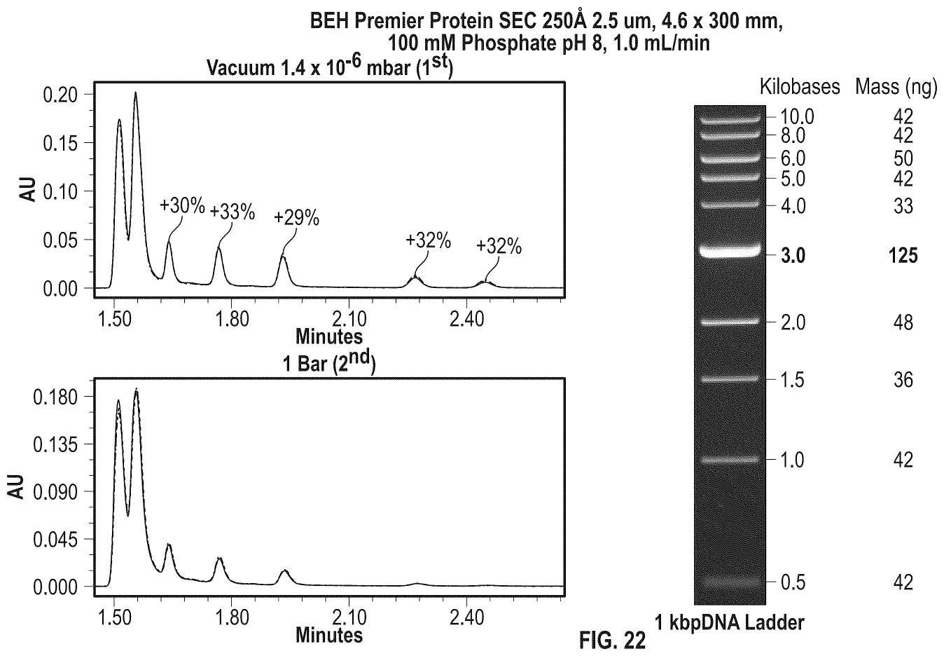

The performance of a chromatography system comprising BEH Premier Protein SEC (250 Å) 2.5 μm particles in a 4.6×300 mm column with a single continuous vacuum jacket was examined. In the first experiment, the vacuum jacket was pressurized to a pressure of approximately 1 bar, simulating diabatic conditions. In the second experiment, the vacuum chamber was evacuated to a pressure of 1.4 nbar. A 1 kilobase pair (1 kbp) DNA ladder was eluted through both columns, and the resulting chromatograms were compared. Each experiment was repeated three times. In each experiment, seven peaks were observed (see FIG. 22). The average results of the relevant properties of the third through seventh peaks are summarized in Table 6.

TABLE 6
1 kbp DNA Ladder Separation Using BEH Premier Protein
SEC 250 Å 2.5 μm Particles

| | | Retention Time | Zeroth | USP Plate | FWHM |
|---|---|---|---|---|---|
| Peak # | | (min) | Moment | Count | (min) |
| 3 | Diabatic | 1.640 | 38688 | 33530 | 0.0213 |
| | Adiabatic | 1.640 | 47070 | 45464 | 0.0181 |
| 4 | Diabatic | 1.769 | 38913 | 27045 | 0.0252 |
| | Adiabatic | 1.767 | 55371 | 36550 | 0.0217 |
| 5 | Diabatic | 1.931 | 26995 | 24196 | 0.0291 |
| | Adiabatic | 1.935 | 54520 | 32184 | 0.0253 |
| 6 | Diabatic | 2.274 | 5949 | 18103 | 0.0394 |
| | Adiabatic | 2.269 | 25961 | 23610 | 0.0346 |
| 7 | Diabatic | 2.455 | 1994 | 12109 | 0.0436 |
| | Adiabatic | 2.447 | 17094 | 21713 | 0.0386 |

Each of peaks 3-7 display similar retention times in both the diabatic and the adiabatic column. The zeroth moment (i.e., relative area of the peak) of adiabatic peaks is generally larger than the corresponding diabatic peak. The adiabatic peaks are also narrower (i.e., have a smaller FWHMV). Taken together, these data indicate that more oligonucleotide is eluting over less time in the adiabatic column. Furthermore, the adiabatic peaks also have largerUSPPlate counts than the diabatic peaks, indicating a more efficient separation.

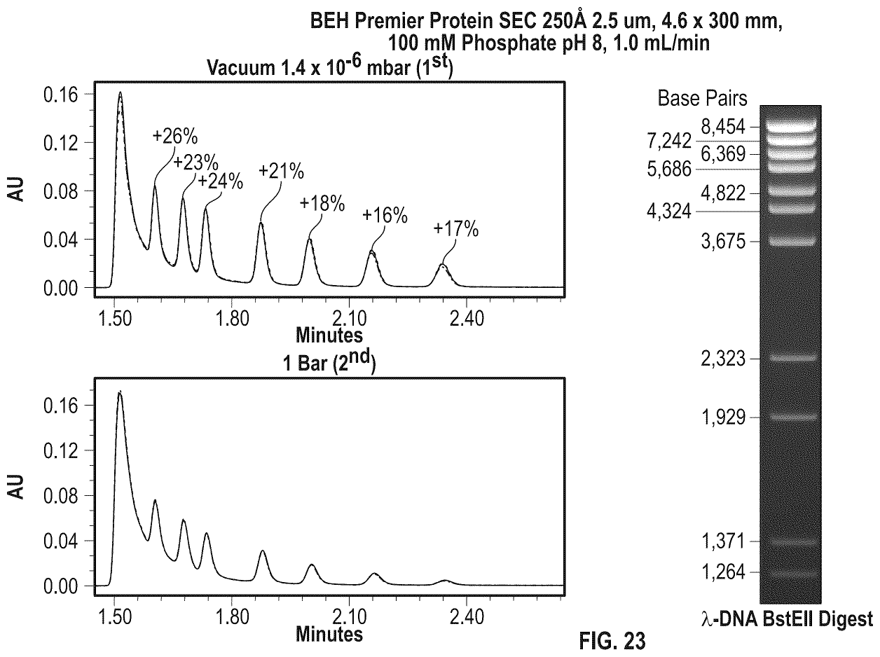

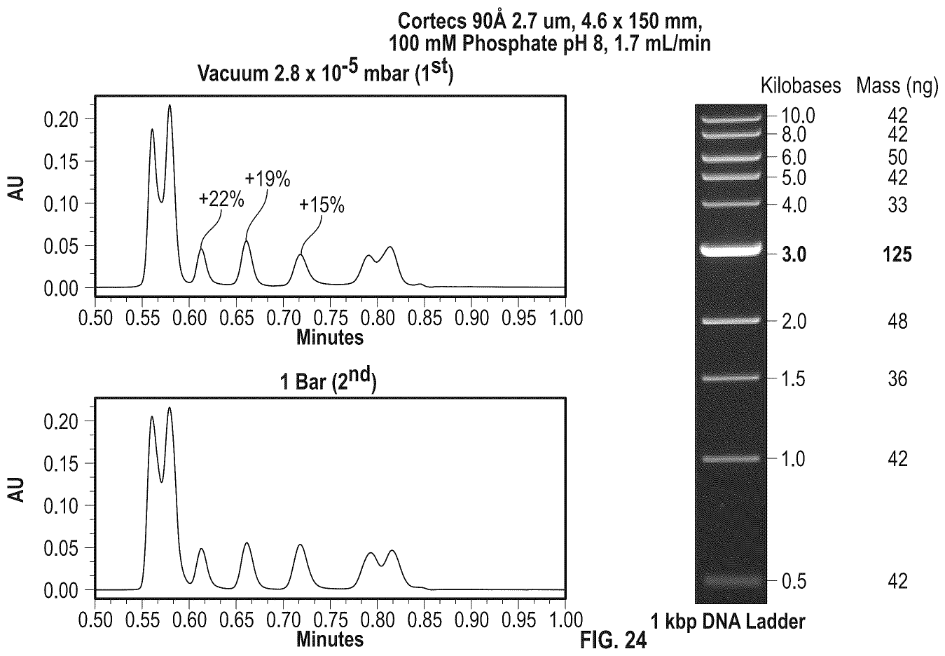

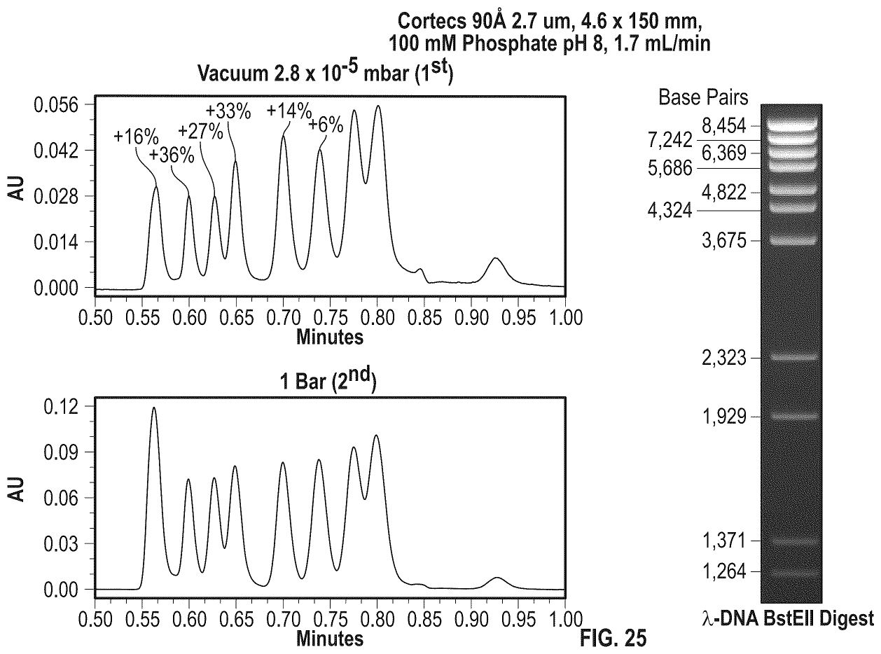

The experiment was then repeated using a λ-DNA BstEII Digest sample in combination with a chromatography system comprising BEH Premier Protein SEC (250 Å) 2.5 μm particles in a 4.6×300 mm column. The adiabatic column had a vacuum pressure of 1.4 nbar, and the diabatic column had a vacuum pressure of 1 bar. Each experiment was repeated three times. In each experiment, eight peaks were observed (see FIG. 23). The average results of the relevant properties of the second through seventh eighth peaks are summarized in Table 7.

TABLE 7
λ-DNA BstEII Digest Separation Using BEH Premier
Protein SEC 250 Å 2.5 μm Particles

| | | Retention Time | Zeroth | USP Plate | FWHM |
|---|---|---|---|---|---|
| Peak # | | (min) | Moment | Count | (min) |
| 2 | Diabatic | 1.605 | 54805 | 32114 | 0.0217 |
| | Adiabatic | 1.604 | 66739 | 39157 | 0.0193 |
| 3 | Diabatic | 1.674 | 73731 | 30767 | 0.0228 |
| | Adiabatic | 1.675 | 56781 | 32895 | 0.0209 |
| 4 | Diabatic | 1.736 | 51316 | 30090 | 0.0241 |
| | Adiabatic | 1.733 | 72927 | 36308 | 0.0216 |
| 5 | Diabatic | 1.879 | 50191 | 25189 | 0.0278 |
| | Adiabatic | 1.875 | 81888 | 30416 | 0.0252 |
| 6 | Diabatic | 2.004 | 34931 | 22867 | 0.0310 |
| | Adiabatic | 1.998 | 70192 | 26917 | 0.0285 |
| 7 | Diabatic | 2.164 | 23458 | 20135 | 0.0357 |
| | Adiabatic | 2.140 | 61578 | 23496 | 0.0330 |
| 8 | Diabatic | 2.343 | 11272 | 17461 | 0.0416 |
| | Adiabatic | 2.338 | 44194 | 20480 | 0.0384 |

Each of peaks 2-8 display similar retention times in both the diabatic and the adiabatic column. The zeroth moment of adiabatic peaks is generally larger than the corresponding diabatic peak, and have a smaller FWHM. Taken together, these data indicate that more oligonucleotide is eluting over less time in the adiabatic column. Furthermore, the adiabatic peaks also have larger USP Plate counts than the diabatic peaks, indicating a more efficient separation.

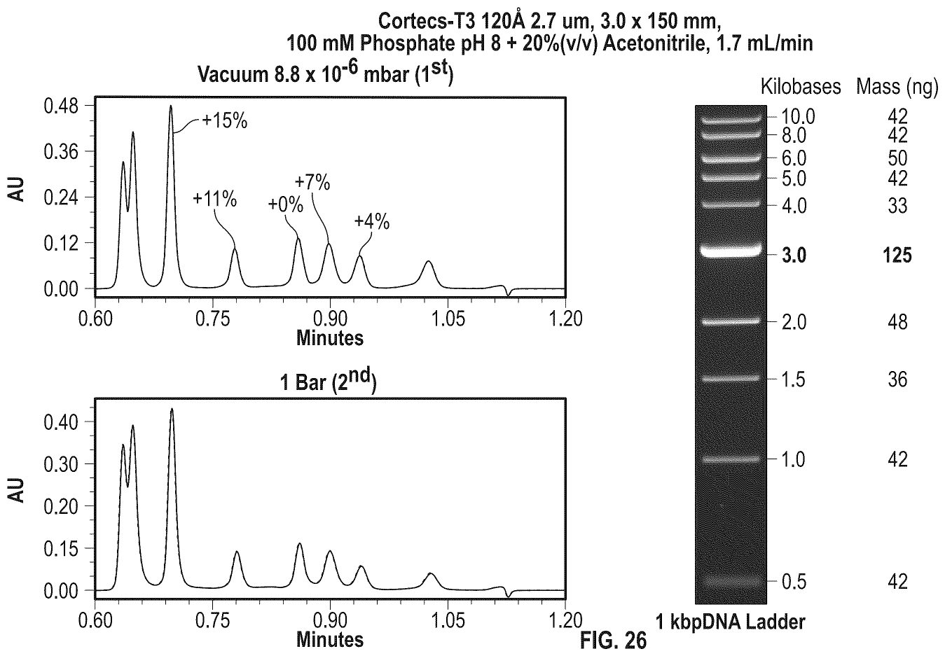

The experiment was then repeated using a 1 kbp DNA ladder sample in combination with a chromatography system comprising a 4.6×150 mm column with (90 Å) 2.7 μm solid-core silica particles (available from Waters Corporation in connection with the Cortecs trade mark) using a mobile phase of phosphate buffered water at pH 8 and a flow rate of 1.7 mL/min. The adiabatic column had a vacuum pressure of 28 nbar, and the diabatic column had a vacuum pressure of 1 bar (see FIG. 24). Seven peaks were observed, the relevant properties of which are summarized in Table 8.

TABLE 8
1 kbp DNA Ladder Separation Using (90 Å) 2.7 μm Particles

| | | Retention Time | Zeroth | USP Plate | FWHM |
|---|---|---|---|---|---|
| Peak # | | (min) | Moment | Count | (min) |
| 1 | Diabatic | 0.561 | 173448 | * | * |
| | Adiabatic | 0.561 | 138891 | 5616 | * |
| 2 | Diabatic | 0.597 | 195213 | * | * |
| | Adiabatic | 0.579 | 163008 | 11181 | * |
| 3 | Diabatic | 0.613 | 44833 | 12425 | 0.0129 |
| | Adiabatic | 0.613 | 40566 | 14706 | 0.0117 |
| 4 | Diabatic | 0.661 | 54281 | 12099 | 0.0142 |
| | Adiabatic | 0.661 | 51436 | 13971 | 0.0130 |
| 5 | Diabatic | 0.718 | 62385 | 10344 | 0.0165 |
| | Adiabatic | 0.718 | 50390 | 9804 | 0.0155 |
| 6 | Diabatic | 0.793 | 55973 | * | * |
| | Adiabatic | 0.791 | 44323 | * | * |
| 7 | Diabatic | 0.816 | 59665 | * | * |
| | Adiabatic | 0.814 | 63075 | * | * |

*Value was not calculable due to insufficient resolution between adjacent peaks.

The experiment was then repeated using a λ-DNA BstEII Digest sample in combination with a chromatography system comprising a 4.6×150 mm column having (90 Å) 2.7 μm solid-core silica particles (column available from Waters Corporation sold in connection with Cortecs trade mark), using a mobile phase of phosphate buffered water at pH 8 and a flow rate of 1.7 mL/min. The adiabatic column had a vacuum pressure of 28 nbar, and the diabatic column had a vacuum pressure of 1 bar. Ten peaks were observed (see FIG. 25). The relevant properties of the first six peaks are summarized in Table 9.

TABLE 9
λ-DNA BstEII Digest Separation Using (90 Å) 2.7 μm Particles

| | | Retention Time | Zeroth | USP Plate | FWHM |
|---|---|---|---|---|---|
| Peak # | | (min) | Moment | Count | (min) |
| 1 | Diabatic | 0.563 | 119857 | 7616 | 0.0155 |
| | Adiabatic | 0.565 | 28261 | 9262 | 0.0144 |
| 2 | Diabatic | 0.599 | 60781 | 12521 | 0.0126 |
| | Adiabatic | 0.600 | 21371 | 16175 | 0.0108 |
| 3 | Diabatic | 0.627 | 63312 | 10751 | 0.0140 |
| | Adiabatic | 0.627 | 21505 | 13547 | 0.0124 |
| 4 | Diabatic | 0.649 | 80230 | 9968 | 0.0151 |
| | Adiabatic | 0.649 | 34743 | 13083 | 0.0131 |
| 5 | Diabatic | 0.700 | 87185 | 10574 | 0.0160 |
| | Adiabatic | 0.700 | 46713 | 11732 | 0.0150 |
| 6 | Diabatic | 0.738 | 96737 | 9770 | 0.0175 |
| | Adiabatic | 0.739 | 46820 | 9655 | 0.0170 |

Each of peaks 1-6 display similar retention times in both the diabatic and the adiabatic column. The adiabatic peaks have a smaller FWHM than the diabatic peaks. Furthermore, the adiabatic peaks also have larger USP Plate counts than the diabatic peaks, indicating a more efficient separation.

The experiment was then repeated using a 1 kbp DNA ladder sample in combination with a chromatography system comprising a 4.6×150 mm column having (120 Å) 2.7 μm solid-core silica particles (available from Waters Corporation in connection with Cortecs trade mark), using a mobile phase of 80% water, 20% acetonitrile with a phosphate buffer at pH 8. The adiabatic column had a vacuum pressure of 8.8 nbar, and the diabatic column had a vacuum pressure of 1 bar. Each experiment was performed twice. In each experiment, eight peaks were observed (see FIG. 26). The average of the relevant properties of peaks 3-7 are summarized in Table 10.

TABLE 10
1 kbp DNA Ladder Separation Using (120 Å) 2.7 μm Particles

| | | Retention Time | Zeroth | USP Plate | FWHM |
|---|---|---|---|---|---|
| Peak # | | (min) | Moment | Count | (min) |
| 3 | Diabatic | 0.698 | 311256 | 24449 | 0.0107 |
| | Adiabatic | 0.697 | 316261 | 26870 | 0.0101 |
| 4 | Diabatic | 0.781 | 71115 | 22101 | 0.0123 |
| | Adiabatic | 0.778 | 80945 | 23764 | 0.0118 |
| 5 | Diabatic | 0.861 | 80018 | 28322 | 0.0122 |
| | Adiabatic | 0.859 | 95288 | 25968 | 0.0124 |
| 6 | Diabatic | 0.900 | 75085 | 20020 | 0.0149 |
| | Adiabatic | 0.898 | 94141 | 21202 | 0.0145 |
| 7 | Diabatic | 0.940 | 45554 | 23529 | 0.0145 |
| | Adiabatic | 0.938 | 68755 | 24189 | 0.0143 |

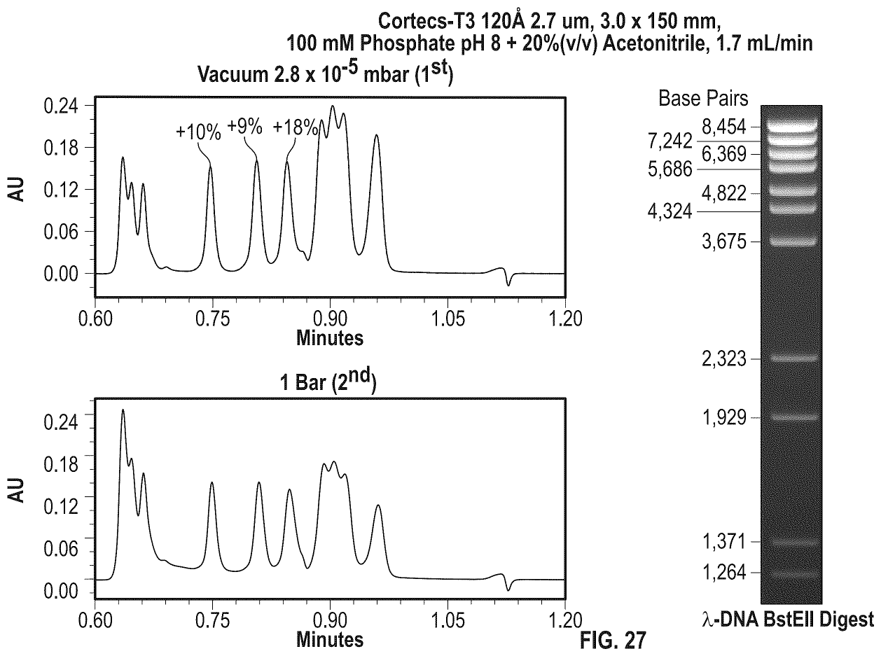

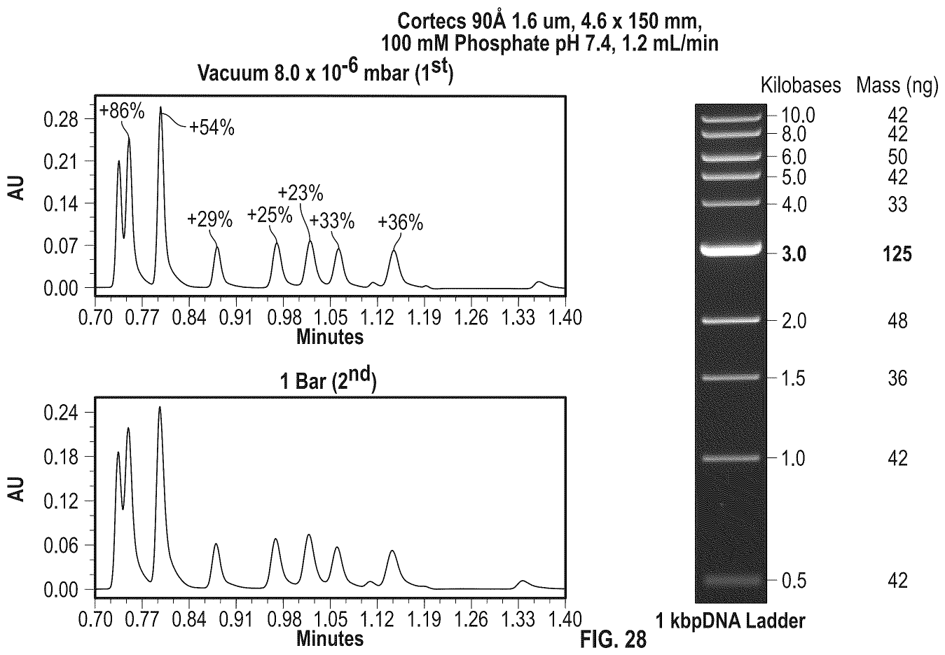

The experiment was then repeated using a λ-DNA BstEII Digest sample in combination with a chromatography system comprising a 4.6×150 mm column having (120 Å) 2.7 μm solid-core silica particles (available from Waters Corporation in connection with the Cortecs trademark), using a mobile phase of 80% water, 20% acetonitrile with a phosphate buffer at pH 8. The adiabatic column had a vacuum pressure of 8.8 nbar, and the diabatic column had a vacuum pressure of 1 bar. Each experiment was performed twice. A multitude of peaks were observed (see FIG. 27). The relevant properties for distinct peaks 1-3 are summarized in Table 11.

TABLE 11
λ-DNA BstEII Digest Separation Using (120 Å) 2.7 μm Particles

| | | Retention Time | Zeroth | USP Plate | FWHM |
|---|---|---|---|---|---|
| Peak # | | (min) | Moment | Count | (min) |
| 1 | Diabatic | 0.749 | 98538 | 23856 | 0.0113 |
| | Adiabatic | 0.747 | 114505 | 25734 | 0.0108 |
| 2 | Diabatic | 0.809 | 103228 | 23046.5 | 0.0125 |
| | Adiabatic | 0.806 | 121831 | 25044 | 0.0120 |
| 3 | Diabatic | 0.848 | 111123 | 17411 | 0.0148 |
| | Adiabatic | 0.845 | 128729 | 24458 | 0.0126 |

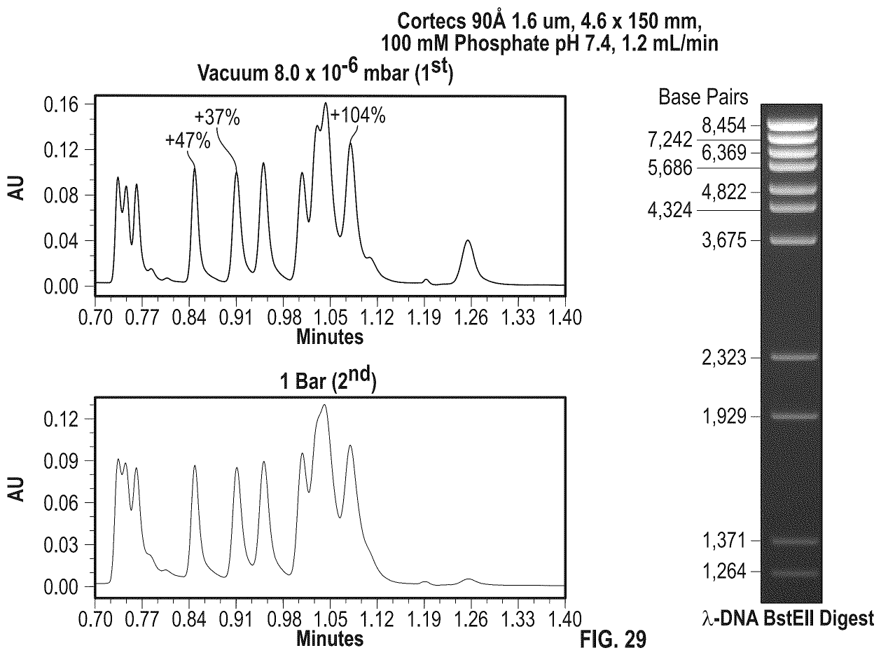

The experiment was then repeated using a 1 kbp DNA ladder sample in combination with a chromatography system comprising a 4.6×150 mm column having (90 Å) 1.6 μm solid-core silica particles (available from Waters Corporation in connection with the Cortecs trade mark) using a mobile phase of phosphate buffered water at pH 7.4 and a flow rate of 1.2 mL/min. The adiabatic column had a vacuum pressure of 8 nbar, and the diabatic column had a vacuum pressure of 1 bar. A multitude of peaks were observed (see FIG. 28). The average of the relevant properties of peaks 1-8 are summarized in Table 12.

TABLE 12
1 kbp DNA Ladder Separation Using (90 Å) 1.6 μm Particles

| | | Retention Time | Zeroth | USP Plate | FWHM |
|---|---|---|---|---|---|
| Peak # | | (min) | Moment | Count | (min) |
| 1 | Diabatic | 0.735 | 119375 | * | * |
| | Adiabatic | 0.736 | 125105 | 32274 | 0.0094 |
| 2 | Diabatic | 0.750 | 200666 | 14208 | 0.0143 |
| | Adiabatic | 0.751 | 196678 | 26834 | 0.0104 |
| 3 | Diabatic | 0.797 | 242739 | 19471 | 0.0129 |
| | Adiabatic | 0.798 | 245438 | 33597 | 0.0101 |
| 4 | Diabatic | 0.880 | 67095 | 21390 | 0.0140 |
| | Adiabatic | 0.882 | 71486 | 27616 | 0.0123 |
| 5 | Diabatic | 0.969 | 79904 | 19086 | 0.0163 |
| | Adiabatic | 0.971 | 82464 | 23762 | 0.0146 |
| 6 | Diabatic | 1.018 | 90108 | 17912 | 0.0176 |
| | Adiabatic | 1.020 | 90968 | 22069 | 0.0159 |
| 7 | Diabatic | 1.060 | 73839 | 18983 | 0.0178 |
| | Adiabatic | 1.062 | 78473 | 25484 | 0.0154 |
| 8 | Diabatic | 1.142 | 77731 | 17187 | 0.0201 |
| | Adiabatic | 1.145 | 83274 | 23706 | 0.0172 |

*Value was not calculable due to insufficient resolution between adjacent peaks.

The experiment was then repeated using a λ-DNA BstEII Digest sample in combination with a chromatography system comprising a 4.6×150 mm column having (120 Å) 1.6 μm solid-core silica particles (available from Waters Corporation in connection with the Cortecs trade mark) using a mobile phase of phosphate buffered water at pH 7.4 and a flow rate of 1.2 mL/min. The adiabatic column had a vacuum pressure of 8 nbar, and the diabatic column had a vacuum pressure of 1 bar. Each experiment was performed twice. A multitude of peaks were observed (see FIG. 29). The relevant properties for distinct peaks 1-3 are summarized in Table 11.

TABLE 13
λ-DNA BstEII Digest Separation Using (90 Å) 1.6 μm Particles

| | | Retention Time | Zeroth | USP Plate | FWHM |
|---|---|---|---|---|---|
| Peak # | | (min) | Moment | Count | (min) |
| 1 | Diabatic | 1.735 | 62559 | * | * |
| | Adiabatic | 0.734 | 56210 | * | * |
| 2 | Diabatic | 0.746 | 62896 | * | * |
| | Adiabatic | 0.746 | 55367 | * | * |
| 3 | Diabatic | 0.762 | 79856 | * | * |
| | Adiabatic | 0.762 | 53261 | 25401 | 0.0108 |
| 4 | Diabatic | 0.849 | 96419 | 20630 | 0.0136 |
| | Adiabatic | 0.848 | 90060 | 30460 | 0.0112 |
| 5 | Diabatic | 0.911 | 95713 | 17628 | 0.0157 |
| | Adiabatic | 0.910 | 94710 | 24546 | 0.0134 |
| 6 | Diabatic | 0.952 | 115186 | 16561 | 0.0170 |
| | Adiabatic | 0.951 | 115667 | 22982 | 0.0145 |
| 7 | Diabatic | 1.009 | 97424 | * | * |
| | Adiabatic | 1.008 | 89455 | * | * |
| 8 | Diabatic | * | * | * | * |
| | Adiabatic | 1.031 | 109537 | * | * |
| 9 | Diabatic | 1.042 | 270249 | 4374 | * |
| | Adiabatic | 1.043 | 186366 | * | * |
| 10 | Diabatic | 1.080 | 191333 | 7400 | 0.0273 |
| | Adiabatic | 1.080 | 160855 | 15775 | 0.0193 |

*Value was not calculable due to insufficient resolution between adjacent peaks.

1. A method of separating a plurality of oligonucleotides, the method comprising:
(a) loading a sample comprising the plurality of oligonucleotides, the plurality of nucleotides including a plurality of a first oligonucleotide and a plurality of a second oligonucleotide, onto a chromatography system;
wherein the chromatography system comprises a chromatography column, a jacket surrounding the liquid chromatography column, and a vacuum chamber formed in an area between the liquid chromatography column and the jacket;
wherein the chromatography column comprises a column body and a plurality of particles, the column body comprising an inlet region, an outlet region, an interior surface, and an exterior surface;
(b) eluting the sample from the chromatography column by contacting the chromatography column with a mobile phase;
wherein the chromatography column is characterized by a flow rate and a pressure differential between the inlet region and the outlet region, wherein the product of the flow rate and the pressure differential is at least 1.5 W/m; and
(c) detecting the plurality of the first oligonucleotide and/or the plurality of the second oligonucleotide in the eluent, wherein the plurality of the first oligonucleotide form at least one distinct peak and wherein the plurality of the second oligonucleotide form at least one distinct peak;
wherein the chromatography column is characterized by a radial temperature gradient of at most 0.1 K and a pressure differential of from the inlet region of the chromatography column to the outlet region of the chromatography column of at least 100 bar.

2. The method of claim 1, wherein the first oligonucleotide comprises DNA or RNA.

3. The method of claim 2, wherein the DNA comprises single stranded DNA (ssDNA) or double stranded DNA (dsDNA), wherein the dsDNA is linear, circular, looped, or supercoiled.

4. (canceled)

5. The method of claim 2, wherein the RNA comprises single stranded RNA (ssRNA) or double stranded RNA (dsRNA), wherein the ssRNA is linear, looped, or circular.

6. (canceled)

7. The method of claim 1, wherein the first oligonucleotide comprises ssRNA, and the second oligonucleotide comprises dsDNA.

8. The method of claim 1, wherein the first oligonucleotide comprises ssDNA, and the second oligonucleotide comprises dsDNA.

9. The method of claim 1, wherein the first oligonucleotide comprises ssRNA, and the second oligonucleotide comprises dsRNA.

10.-16. (canceled)

17. A method of separating DNA, the method comprising:
(a) loading a sample comprising a plurality of dsDNA molecules and a plurality of ssDNA molecules onto a chromatography system;
wherein the chromatography system comprises a chromatography column, a jacket surrounding the liquid chromatography column, and a vacuum chamber formed in an area between the liquid chromatography column and the jacket;
wherein the chromatography column comprises a column body and a plurality of particles, the column body comprising an inlet region, an outlet region, an interior surface, and an exterior surface;
(b) eluting the sample from the chromatography column by contacting the chromatography column with a mobile phase;
wherein the chromatography column is characterized by a flow rate and a pressure differential between the inlet region and the outlet region, wherein the product of the flow rate and the pressure differential is at least 1.5 W/m; and
(c) detecting the plurality of dsDNA and/or the plurality of ssDNA in the eluent, wherein the plurality of dsDNA forms at least one distinct peak, and the plurality of ssDNA forms at least one distinct peak;
wherein the chromatography column is characterized by a radial temperature gradient of at most 0.1 K and a pressure differential from the inlet region to the outlet region of at least 100 bar.

18. (canceled)

19. The method of claim 1, wherein the mobile phase comprises water and one or more salts; wherein the mobile phase does not comprise acetonitrile.

20. The method of claim 1, wherein the mobile phase comprises water, acetonitrile, and one or more salts.

21. (canceled)

22. The method of claim 20, wherein the acetonitrile is less than 30%_{v/v} of the mobile phase.

23. (canceled)

24. The method of claim 1, wherein the eluting in step (b) is characterized by a k₁ of less than 0.35.

25.-26. (canceled)

27. The method of claim 1, wherein the eluting in step (b) is performed in from about 0.6 minutes to about 10 minutes.

28.-29. (canceled)

30. The method of claim 1, wherein the plurality of particles comprise an organic material.

31. The method of claim 1, wherein the plurality of particles comprise a material with a thermal conductivity of at most 2 W/m·K.

32.-52. (canceled)

53. The method of claim 1, wherein the chromatography column is characterized by an effective thermal conductivity of at most 2 W/m·K.

54.-55. (canceled)

56. The method of claim 1, wherein the vacuum chamber comprises a pressure of at most 1 mbar.

57. (canceled)

58. The method of claim 1, wherein the vacuum chamber comprises an inert gas, the inert gas selected from argon, krypton, xenon, carbon dioxide, and sulfur hexafluoride.

59.-64. (canceled)

65. The method of claim 1, wherein the at least one distinct peak is characterized by an increase in the USP plate count compared to a chromatography column not including a vacuum chamber.

66.-68. (canceled)

69. The method of claim 1, wherein the at least one distinct peak is characterized by a decrease in full-width-at-half-max compared to a chromatography column not including a vacuum chamber.

69.-72. (canceled)
